(19) United States
(12) Patent Application Publication
(10) Pub. No.: US 2024/0086460 A1
(43) Pub. Date: Mar. 14, 2024

(54) WHOLE SLIDE IMAGE SEARCH

(71) Applicants: Genentech, Inc., South San Francisco, CA (US); Roche Molecular Systems, Inc., Pleasanton, CA (US); Hoffmann-La Roche Inc., Little Falls, NJ (US)

(72) Inventors: Ido BEN SHAUL, Ra'anana (IL); Marta CANAMERO, Starnberg (DE); Ofir ETZ HADAR, Hod Hasharon (IL); Jacob GILDENBLAT, Ra'anana (IL); Fang-Yao HU, San Mateo, CA (US); Eldad KLAIMAN, Starnberg (DE)

(21) Appl. No.: 18/516,212

(22) Filed: Nov. 21, 2023

Related U.S. Application Data
(63) Continuation of application No. PCT/US2022/031845, filed on Jun. 1, 2022.
(60) Provisional application No. 63/195,883, filed on Jun. 2, 2021.

(51) Int. Cl. G06F 16/583; G06F 16/51; G06F 16/532; G06F 16/535; G06F 16/538; G06T 7/11

(52) U.S. Cl. CPC G06F 16/583; G06F 16/51; G06F 16/532; G06F 16/535; G06F 16/538; G06T 7/11

(57) ABSTRACT

In one embodiment, a method includes indexing a whole slide image dataset to generate one or more dataset embeddings corresponding to one or more respective regions of one or more whole slide images. Each dataset embedding includes a feature vector mapping the respective region to a feature embedding space. The method includes accessing a query image and generating an embedding for the query image that includes a feature vector mapping the query image to the feature embedding space. The method includes identifying result tiles by comparing the embedding for the query image to one or more of the dataset embeddings. The comparison is based on one or more distances between the embedding for the query image and the one or more of the dataset embeddings in the feature embedding space. The method includes generating a user interface including a display of the result tiles.

### CROSS-REFERENCE TO RELATED APPLICATION

The present application is a bypass continuation application of International Application PCT/US2022/031845, filed Jun. 1, 2022, which claims the benefits of U.S. Provisional Application No. 63/195,883, filed Jun. 2, 2021, the entire content of each of which are incorporated herein by reference in their entirety for all purposes.

### TECHNICAL FIELD

This disclosure generally relates to tools for analyzing and searching for digital pathology images.

### BACKGROUND

Whole Slide Images (WSI) result from scans of images of samples or from digital-native scans. A scan, and the corresponding WSI, is often very large, for example 100,000 pixels by 100,000 pixels in each of several color channels, making it difficult to efficiently analyze WSI on a holistic level using traditional computational methods. Current approaches to handle the large formats of WSI include segmenting the WSI into smaller portions and performing parallel analysis using multiple processors or otherwise distributed processing.

A pathologist or other trained specialist will often evaluate a single WSI for evidence of abnormalities in the depicted tissue. Labeling for WSI tends to refer to the entire image and not, for example, to a specific portion of an image. For example, a pathologist may identify a tissue abnormality (e.g., a tumor) in an image of a lung and label the image as “abnormal.” In most cases, however, the pathologist will not annotate the image to specify where in the image the tissue abnormality appears. This “all or nothing” labelling style is less useful for identifying features common to a set of whole slide images, as even when a WSI is labeled, the location of the feature is typically not labelled. Instead, if a pathologist wishes to compare a specific feature to a library of WSI, they must rely on this rudimentary labelling, or on their own recall, to select the appropriate WSI. Ultimately, they must manually identify the feature within the appropriate WSI. This greatly limits the scope of comparison that can be performed across WSI, greatly decreasing the chances that a pathologist will be able to effectively identify and compare uncommon features.

Accordingly, a desire exists for systems to enable pathologists and other users to query a set of whole slide images using an arbitrary query image to identify similar features or components of the set of whole slide images. In addition, a desire exists for tools to facilitate the generation and sharing of reports relating to results from said query.

### SUMMARY OF PARTICULAR EMBODIMENTS

In particular embodiments, a computer-implemented method includes indexing a whole slide image dataset to generate one or more dataset embeddings corresponding to one or more respective regions of one or more whole slide images. Each dataset embedding includes a feature vector mapping the respective region to a feature embedding space. The computer accesses a query image and generates an embedding for the query image that includes a feature vector mapping the query image to the feature embedding space. The computer identifies result tiles by comparing the embedding for the query image to one or more of the dataset embeddings. The comparison is based on one or more distances between the embedding for the query image and the one or more of the dataset embeddings in the feature embedding space. The computer generates a user interface including a display of the result tiles. In some embodiments, identifying the result tiles includes identifying some of the dataset embeddings based on the embedding for the query image and retrieving the one or more respective regions corresponding to one or more of the dataset embeddings. In some embodiments, identifying the dataset embeddings based on the embedding for the query image includes identifying dataset embeddings that are within a threshold distance of the embedding for the query image in the feature embedding space. In embodiments, identifying the dataset embeddings based on the embedding for the query image includes identifying a threshold number of the dataset embeddings ordered based on distance to the embedding for the query image in the feature embedding space. In some embodiments, the computer receives a user input corresponding to one or more of the result tiles, the user input comprising a marking of one or more of the result tiles. The computer receives a user input corresponding to a weighting associated with the one or more marked result tiles and generates an object filter based on the one or more marked result tiles and the user input corresponding to the weighting associated with the one or more marked result tiles. The computer augments the embedding for the query image based on a representation of the object filter. The computer identifies a second set of result tiles by comparing the embedding for the augmented query image to one or more of the dataset embeddings. The computer updates the user interface to display the second set of result tiles. In some embodiments, applying the generated object filter to the one or more dataset embeddings includes comparing the one or more dataset embeddings to the generated object filter in the feature embedding space. In some embodiments, the computer receives, from a user device, a user input to save the generated object filter and stores the generated object filter in association with a record for one or more users of the user device. In some embodiments, the computer receives, from a user device, a user input to share the generated object filter with one or more other users and stores the generated object filter in association with a record for one or more other users.

In particular embodiments, indexing the whole slide image dataset includes, for each of a set of whole slide images: segmenting the whole slide image into a set of tiles; generating, using an embedding network, a feature vector corresponding to each tile of the set of tiles that maps the tile to the feature embedding space; and storing the feature vector in association with the corresponding tile and whole slide image. In particular embodiments, accessing the query image includes receiving the query image from a user device; receiving a resource locator or unique identifier corresponding to the query image; or receiving a specification of a region of a whole slide image. In some embodiments, the query image corresponds to a whole slide image. The computer can index the whole slide image corresponding to the query image to generate one or more additional dataset embeddings corresponding to one or more respective regions of the whole slide image. The computer adds the whole slide image corresponding to the query image to the whole slide image dataset. In some embodiments, the computer receives a user input corresponding to one or more of the result tiles, the user input indicating a relevance of the one or more of the result tiles to the query image. The computer modifies a weighting of the one or more indicated results based on the user input. The computer identifies a second set of result tiles by comparing the embedding for the query image to one or more of the dataset embeddings based on the modified weighting. The computer updates the user interface to display the second set of result tiles. In embodiments, the computer receives a user input corresponding to one or more of the result tiles, the user input indicating a relevance of the one or more of the plurality of result tiles to the query image. The computer computes an average embedding of the relevant search results. The computer identifies a second set of result tiles by comparing the average embedding of the relevant search results to one or more of the dataset embeddings. The computer updates the user interface to display the second set of result tiles. In some embodiments, the computer receives, from a user device, a user input corresponding to a first result tile of the result tiles. The computer identifies a first whole slide image corresponding to the first result tile and updates the user interface to display the first whole slide image. In some embodiments, the computer further identifies metadata associated with the first whole slide image or the first result tile and includes the metadata in the user interface including the display of the first whole slide image. The metadata can include information regarding the first result tile, the first whole slide image, or a source of the first whole slide image. In some embodiments, the computer identifies a set of whole slide images corresponding to the result tiles. The computer identifies a set of sources of the whole slide images. The computer updates the user interface to display a report of information corresponding to the sources of the whole slide images. In embodiments, the information corresponding to the sources includes conditions diagnosed in the sources or known outcomes associated with the sources. In embodiments, the computer identifies, based on the result tiles, a respective location of one or more features captured in the query image in the whole slide images corresponding to the result tiles and updates the user interface to display a report of the identified respective locations.

The embodiments disclosed herein are only examples, and the scope of this disclosure is not limited to them. Particular embodiments may include all, some, or none of the components, elements, features, functions, operations, or steps of the embodiments disclosed herein. Embodiments according to the invention are in particular disclosed in the attached claims directed to a method, a storage medium, a system and a computer program product, wherein any feature mentioned in one claim category, e.g., method, can be claimed in another claim category, e.g., system, as well. The dependencies or references back in the attached claims are chosen for formal reasons only. However any subject matter resulting from a deliberate reference back to any previous claims (in particular, multiple dependencies) can be claimed as well, so that any combination of claims and the features thereof are disclosed and can be claimed regardless of the dependencies chosen in the attached claims. The subject matter that can be claimed includes not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example method for whole slide image search.

FIG. 2 illustrates an example method for refinement by example selection in whole slide image search.

FIG. 3 illustrates an example method for object filter generation in whole slide image search.

FIG. 4 illustrates an example method for whole slide output generation in whole slide image search.

FIG. 5 illustrates an example method for dataset output generation in whole slide image search.

FIGS. 6A and 6B illustrate an example user interface for receiving query input for whole slide image search.

FIG. 6C illustrates an example field displaying an image and adjustable field of view selector.

FIGS. 7A and 7B illustrate an example user interface including tile results.

FIG. 7C illustrates an example display that shows a query image

FIGS. 8A and 8B illustrate an example user interface including whole slide results.

FIG. 9A illustrates an example user interface including refinement by example selection.

FIG. 9B illustrates example positively-marked and negatively-marked tiles.

FIGS. 10A and 10B illustrate example user interfaces including object filter generation.

FIG. 10C illustrates an example image viewer.

FIGS. 11A and 11B illustrate a second example user interface for receiving query input for whole slide image search.

FIG. 12 illustrates an example user interface including additional output.

FIG. 13 illustrates an example whole slide image search system.

FIG. 14 illustrates an example artificial neural network.

FIG. 15 illustrates an example computer system.

### DESCRIPTION OF EXAMPLE EMBODIMENTS

Analysis of whole slide images (WSI) is a labor-intensive process that requires highly specialized individuals with the knowledge and dexterity to the review the WSI, recognize and identify abnormalities, classify the abnormalities, label the WSI, and potentially render diagnosis of the tissue. Additionally, because WSI are used for a wide array of tissue types, persons with the knowledge and skill to identify abnormalities must be further specialized in order to provide accurate analysis and diagnosis. The problem is compounded in the context of comparing features of one WSI to another, or in identifying similar or related features across multiple WSIs. For example, it would be an extremely time-consuming task for an individual to identify a feature of a subject WSI and look for other WSI exhibiting the same feature, as in identifying possible results to a search based on the subject WSI. Because of the labor- and knowledge-intensive nature of the work, WSIs are candidates for automation of certain functions, including search. However, the large size of WSIs renders typical techniques ineffective, slow, and expensive. It is not practical to perform standard image recognition and deep learning techniques, which require analysis of multiple rounds of many samples of WSIs to increase accuracy. Once again, the problem is exacerbated in the context of matching arbitrary features to features of the WSIs. The techniques described herein are directed to solving the problem of image search and recognition in WSI and enable the development of novel data analysis and presentation techniques that previously could not be performed with WSI due to the well-documented limitations.

The systems disclosed herein can efficiently generate embeddings of a large dataset of WSIs based on distinct regions of the dataset and prepare the dataset for comparison against an arbitrary query image. The system disclosed herein can further provide for user-driven customization and refinement of image search to improve the quality of results in a manner based on the needs of the user at any given time. A whole slide image search system according to the description herein enables users to search a large dataset of whole slide images based on a selected query image or field of view. The query results include image tiles or fields of views from the whole slide image database that are determined to be similar to the query image as described herein. The user can then interact with the retrieved images, further explore, and choose to refine the search or generate specified output visualizations and reports.

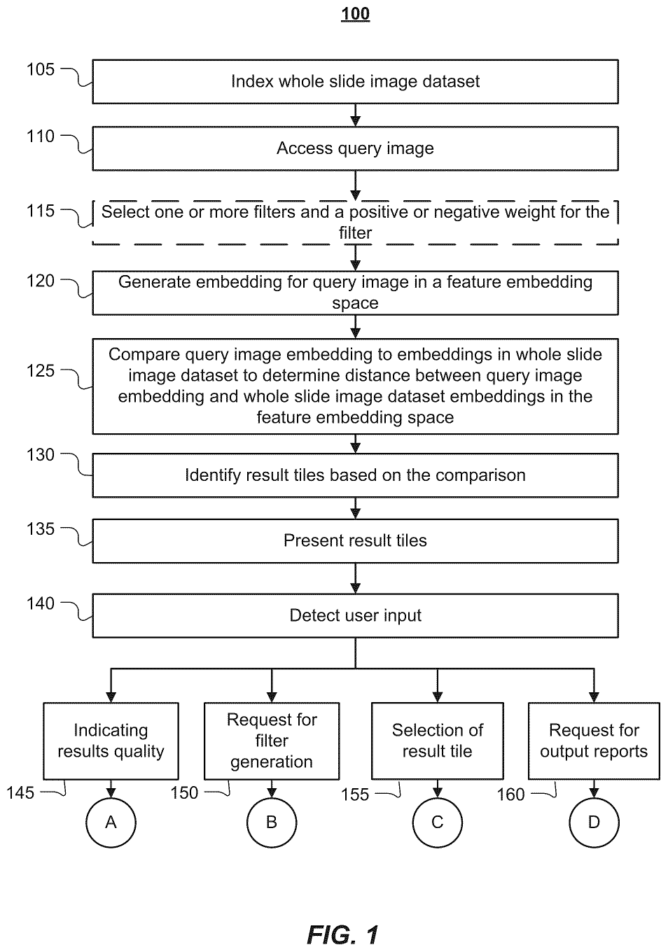

FIG. 1 illustrates an example method for whole slide image search. The method beings at step 105, where the whole slide image search system indexes a dataset of whole slide images. The index that is created includes each whole slide image, a set of tiles segmented from each whole slide image, and embeddings generated for each of the tiles. Additionally, metadata and other information describing each whole slide image and tile thereof can be attributed to the whole slide image dataset. In particular embodiments, the dataset is stored across multiple databases communicatively coupled to the whole slide image search system. The databases can, in particular embodiments, be organized according to the type of data stored, the location of the source data or expected requests for access, or other data organization protocols.

The whole slide images for the dataset can be accessed by the whole slide image search system from a database of whole slide image that have been collected by one or more users of the whole slide image search system or by other systems that are in communication with the whole slide image search. The images may have been collected for a variety of purposes and, besides the occasional retrieval for historical review or verification, are sitting idle in databases. The whole slide image search system can repurpose the whole slide images into an active dataset that is easier to explore by indexing the images.

To index the whole slide images for the data, the whole slide image search system segments each whole slide image into multiple tiles. Each whole slide image is expected to be significantly larger than standard images, and much larger than would normally be feasible for standard image recognition and analysis (e.g., on the order of 100,000 pixels by 100,000 pixels). To facilitate analysis, the whole slide image search system segments each whole slide image into tiles. The size and shape of the tiles are uniform for the purposes of analysis, but the size and shape can be variable. In some embodiments, the tiles can overlap to increase the opportunity for image context to be properly analyzed by the whole slide image search system. To balance the work performed with accuracy, it may be preferable to use non-overlapping tiles. Additionally, segmenting the image into tiles can involve segmenting the image based on a color channel or dominant color associated with the image.

Next, the whole slide image search system, for example using a tile embedding module, generates an embedding for each tile using an embedding network. As described herein, the embeddings can include unique representations of the tiles that preserve some information about the content or context of the tiles. The tile embeddings can also be derived from a mapping of the tiles into a corresponding tile embedding space or feature embedding space. The resulting embedding can be considered representative of the features shown in the tile. Within the feature embedding space, tiles in spatial proximity may be considered similar, while distance between tiles in the feature embedding space may be indicative of dissimilarity. For example, based on the embeddings for each tile generated using the embedding network, tiles that depict similar subject matter or have similar visual features will be positioned with less inter-embedding distance in the feature embedding space than tiles that depict different subject matter or have dissimilar visual features. The tile embeddings can be represented as feature vectors that map the tile to the feature embedding space. The whole slide image search system can generate tile embeddings using a neural network (e.g., a convolutional neural network) to generate a feature vector that represents each tile of the image, also referred to as an embedding network. The embedding network receives tiles (e.g., images) as input and produces embeddings (e.g., vector representations) as output. In particular embodiments, the tile embedding neural network can be based on the ResNet image network trained on a dataset based on natural (e.g., non-medical) images, such as the ImageNet dataset. By using a non-specialized tile embedding network, the whole slide image search system can leverage known advances in efficiently processing images to generating embeddings. Furthermore, using a natural image dataset allows the embedding neural network to learn to discern differences between tile segments on a holistic level and increases the sophistication of available training data.

In other embodiments, the tile embedding network can be an embedding network customized to handle large numbers of tiles of large format images, such as whole slide images. Additionally, the tile embedding network can be trained using a custom dataset. For example, the tile embedding network can be trained using a variety of samples of whole slide images or even trained using samples relevant to the subject matter for which the embedding network will be generating embeddings (e.g., scans of particular tissue types). Training the tile embedding network using specialized or customized sets of images can allow the tile embedding network to identify finer differences between tiles which can result in more detailed and accurate distances between tiles in the feature embedding space at the cost of additional time to acquire the images and the computational and economic cost of training multiple tile generating networks for use by the whole slide image search system in different contexts. The whole slide image search system can select from a library of tile embedding networks based on the type of images being indexed (or later, searched).

Tile embeddings can be generated from a deep learning neural network using visual features of the tiles. Each tile can be treated as an independent component for the purpose of embedding generation. In this way, tiles that are near each other in a given whole slide image will not necessarily be near each other in the embedding space. Instead, the embedding space can be focused on grouping tiles exhibiting similar features.

Tile embeddings can be further generated using contextual information associated with the tiles or from the content shown in the tile. For example, a tile embedding can include one or more features that indicate and/or correspond to a size of depicted objects (e.g., sizes of depicted cells or aberrations) and/or density of depicted objects (e.g., a density of depicted cells or aberrations). Size and density can be measured absolutely (e.g., width expressed in pixels or converted from pixels to nanometers) or relative to other tiles from the same digital pathology image, from a class of digital pathology images (e.g., produced using similar techniques or by a single digital pathology image generation system or scanner), or from a related family of digital pathology images. Furthermore, tiles can be classified prior to the whole slide image search system generating embeddings for the tiles such that the embeddings consider the classification when preparing the embeddings.

In particular embodiments, tile locality can be considered. When generating tile embeddings, one feature provided to the embedding network can be the identification of tiles near a subject tile, with the goal being to account, in some way, for the position of the tile within a whole slide image. This may allow the embedding network to automatically infer the context of the tile. Other additional features can also be added in addition to the visual components of the tile and the locality. For example, the identification of the slide can be used to add the additional context of the global identity of the tiles. This may have the effect of smoothing the locality of results. As another example, temporal aspects of a slide can be incorporated into the tile embeddings. Slides that come from the subject (e.g., patient) at different points in time can be grouped, showing the progression of a condition exhibited therein. Additionally, the embedding network may be configured to automatically group each user's slides. Thus, a given user (e.g., a researcher) can more easily find results related to their slides. These additional features can also be added by appending features to the feature vector representation generated by the embedding network of the whole slide image search system. For example, the tile embedding can be generated as previously described, with information about locality or subject history encoded into the embedding for comparison purposes during a search query. Distance can also be calculated using multiple distinct functions, with the type and quality of results potentially changing as the distance function changes.

For consistency, the whole slide image search system may produce embeddings of a predefined size (e.g., vectors of 512 elements, vectors of 2048 bytes, etc.). The whole slide image search system can produce embeddings of various and arbitrary sizes. The whole slide image search system can adjust the sizes of the embeddings based on user direction or can be selected, for example, to optimize computation efficiency, accuracy, or other parameters. In particular embodiments, the embedding size can be based on the limitations or specifications of the deep learning neural network that generated the embeddings. Larger embedding sizes can be used to increase the amount of information captured in the embedding and improve the quality and accuracy of results, while smaller embedding sizes can be used to improve computational efficiency.

In particular embodiments, the whole slide image search dataset can be augmented or annotated to reference canonical example of histopathological artifacts. For example, conditions may occur commonly in certain types of tissues or situations. The whole slide image search system can be provided many examples of the condition, which may then be embedded. Searches that match one of the canonical examples can be specifically flagged to facilitate the user quickly identifying their searched feature as corresponding to one of these designated examples.

After the whole slide image dataset is indexed at step 105, at step 110, the whole slide image search system accesses a query image. As described in further detail herein, the whole slide image search system can receive the query image in a variety of ways. For example, the whole slide image search system can be made available to a user through a thin client or web browser executing on a user device. The user device can be a computer used by a pathologist or clinician connected via one or more networks to the whole slide image search. The user can select and upload a query image from the user device to the whole slide image search system. As another example, the whole slide image search system can be communicatively coupled to a database of whole slide images. The user can instruct the whole slide image search system to access one of the whole slide images and can specify a particular region of the whole slide image to use as the query image. In particular embodiments, the region can be selected based on a current field of view of the user or a selected boundary. The region may align with a pre-segmented tile of the whole slide image but is not restricted to such a boundary. As another example, the whole slide image search system can receive the whole slide image from a whole slide image generation system or one or more components thereof. There may be restrictions on the types of images that can be submitted by a user. For example, while a subset of a whole slide image can be used (e.g., a selection of a particular tile or a region of the whole slide image that is approximately equivalent to a tile), a whole slide image itself may not be allowed as the system would be unable to process the image efficiently. Moreover, the whole slide image search system is capable of identifying similarities across features, and a full whole slide image would lose the specificity of these features.

At step 115, the whole slide image search system may receive a selection of one or more object filters associated with the results and a positive or negative weight for the object filter. An object filter acts as a way of refining the search results by providing additional information about the desired results. For example, an object filter can be trained to favor results from a particular type of tissue, results showing a particular abnormalities in a features, etc. Similarly, an object filter can be trained to detect aberrations in an image, such as an artifact introduced in the creation of a whole slide image or a tile. The artifact can be identified and treated as an object associated with or to be identified within the image. A negative weight applied to this concept can exclude the aberrations from the search results. Therefore, although object filters refer to the filtering of a particular object or class of object, the object filter can be trained to identify, and filter based on any concept which can be graphically represented in an image or be otherwise associated with the image (e.g., based on metadata stored with or associated with the image). Object filters function as a form of an “advanced” search, where search using a query image alone is similar to a more basic or naïve search. In some cases, the user may not have particular object filters trained and ready for use or may not wish to use an object filter at this time. Therefore this step is considered optional. As described herein, object filters can be trained from search results after an initial query is performed, allowing for results to be refined over time.

At step 120, the whole slide image search system generates an embedding for the query image using an embedding network or other artificial neural network. The embedding for the query image can be generated using the same principles as the embeddings for the tiles included in the whole slide image dataset. Moreover, the same embedding network can be used to ensure a consistent mapping of the query image to the feature embedding space.

If the whole slide image search system determines that the input image is, or is derived from, a whole slide image, the whole slide image search system may automatically add the query image to the whole slide image dataset, subject to establishing the proper use of the whole slide image. To facilitate adding the query image to the whole slide image dataset, the whole slide image search system may prompt the user to provide additional metadata or prompt the user to direct the whole slide image search system to a location on the network or a user device where the appropriate metadata is stored. As described herein, the metadata can optionally be used to limit or refine search results and may also be used to provide additional contextual information regarding result tiles upon request of the searching user.

At step 125, the whole slide image search system compares the embedding generated from the query image to whole slide image dataset. The whole slide image search system compares the embedding generated from the query image to embeddings generated from one or more of the whole slide images from the dataset. As described herein, the embeddings can be described as having a representation of a location within a feature embedding space. Comparing the embeddings can include determining a distance in the feature embedding space between the locations of the embedding generated from the query image and the embeddings generated from the whole slide image search dataset. Each of the embeddings can represent a tile extracted from the whole slide image, so comparing the embeddings results in comparing the tiles from the images of the whole slide image dataset to the embedding from the query image. The meaning of the distance is dependent on the information used to generate the embedding. For example, where the embedding includes only visual information for a single tile, distance will correlate with visual similarity. Where the embedding includes additional information, such as inter-tile relationships or locality, distance will include measures for those features as well.

Where the user has specified a distance calculation methodology, that calculation can be used by the whole slide image search system. Additionally, when the user has specified one or more object filters to be used, they may be applied when comparing the embedding generated from the query image to the embeddings generated from the whole slide image search. In particular, positively-weighted object filters are given a positive weight for the distance calculation, favoring embeddings made “close” by the object filter. Negatively-weighted object filters are given a negative weight for the distance calculation, disfavoring embeddings made “close” by the object filter. Where the object filter is represented as an embedding generated from a support vector machine hyperplane, incorporating the object filter can include comparing each of the whole slide image search dataset to the object filter embedding as well.

As described above, whole slide images are often very large and large numbers of tiles can be produced from even a small number of whole slide images. It is expected, for example, for even a dataset of hundreds of whole slide images to include millions of tiles and tile embeddings. Therefore, the query image embedding must be compared against these millions of tiles in order to determine which are the most similar. In particular embodiments, the task of comparing the tile embeddings of the whole slide image dataset to the query image embedding can be distributed across multiple agents of the whole slide image search system. For example, this task can be distributed across the group of agents, with each agent comparing a subset of the tile embeddings of the whole slide image dataset to the query image and provide a set of top matches. A master or control subsystem of the whole slide image search system can then compare the top matches across the group of agents to identify the top overall results (e.g., the embeddings with the least distance to the embedding for the query image in the embedding space). Where parallel access will be required by multiple agents, the embeddings are each stored in independent files within the database(s) of the whole slide image search system. Additionally, in particular embodiments, the embeddings of the whole slide image search dataset can be further compressed through a variety of mathematical treatments to reduce storage size and control search time by limiting the number of individual comparisons that must be made. Compression methods can include averages of the coordinates of the feature vector presentation, singular value decomposition, or other computational methods.

At step 130, the whole slide image search system identifies result tiles based on the comparison. The identified tiles will correlate to those embeddings identified as being most likely to be responsive to the query image. As an example, the whole slide image search system can identify the tiles corresponding to the nearest embeddings of the whole slide image data set as result tiles.

At step 135, the whole slide image search system can present the result tiles to the user of the whole slide image search system. In certain embodiments, that can involve presenting a subset of the result tiles in an interactive user interface on a client device. The subset that is presented to the user can, in particular embodiments, be ordered based on the distance between the embedding corresponding to each result tile and the embedding generated from the query image in the feature embedding space. In particular embodiments, the ranking can be provided to the user. In particular embodiments, the subset of the result tiles can include all tiles with an embedding within a defined threshold distance of the embedding generated for the query image in the feature embedding space. In particular embodiments, the subset of the result tiles can include the top N results, where N is a system defined or user defined number of results, and the top N results represents the N result tiles corresponding to embeddings with the lowest distance to the embedding generated for the query image in the feature embedding space ordered based on the distance. In this example, N represents the threshold number of results to be returned or provided. The interactive user interface can include a variety of interactive elements to enable the user to engage with further functions of the whole slide image search system. The whole slide image search system can monitor for user input relating to those additional functions.

At step 140, the whole slide image search system monitors for and detects a user input regarding the quality of the results, such as a selection of one or more of the results as falling into one or more categories, e.g., being a “positive” result or a “negative” result, being a relevant result or an irrelevant result, etc. In this context, a positive result may correlate with a result of the kind or similar to that which the user expected or wanted to receive. Similarly, a negative result may correlate with a result that is dissimilar to what the user expected or wanted to receive. The indication of a result as positive can correspond with a user desiring to receive additional results like the indicated result. The indication of a result as a negative result can correspond with a user desired to not receive additional results like the indicated result. When a user input is detected, the one or more computing systems of whole slide image search system determine the nature of the user input and the method continues.

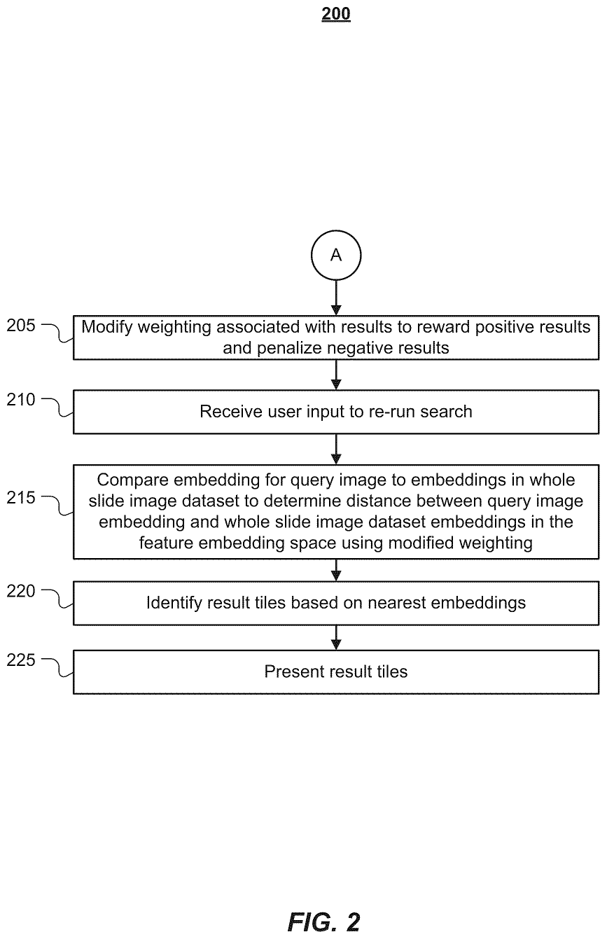

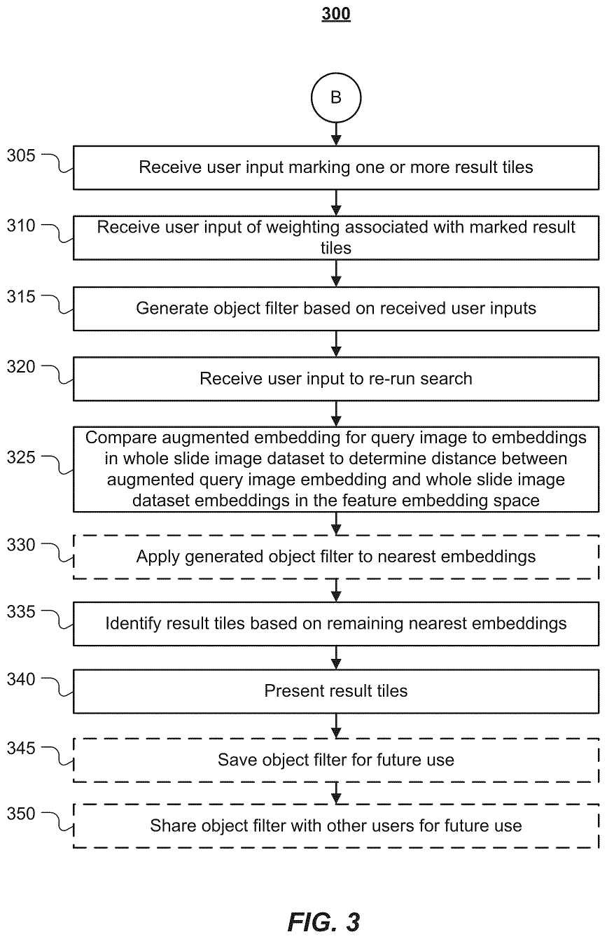

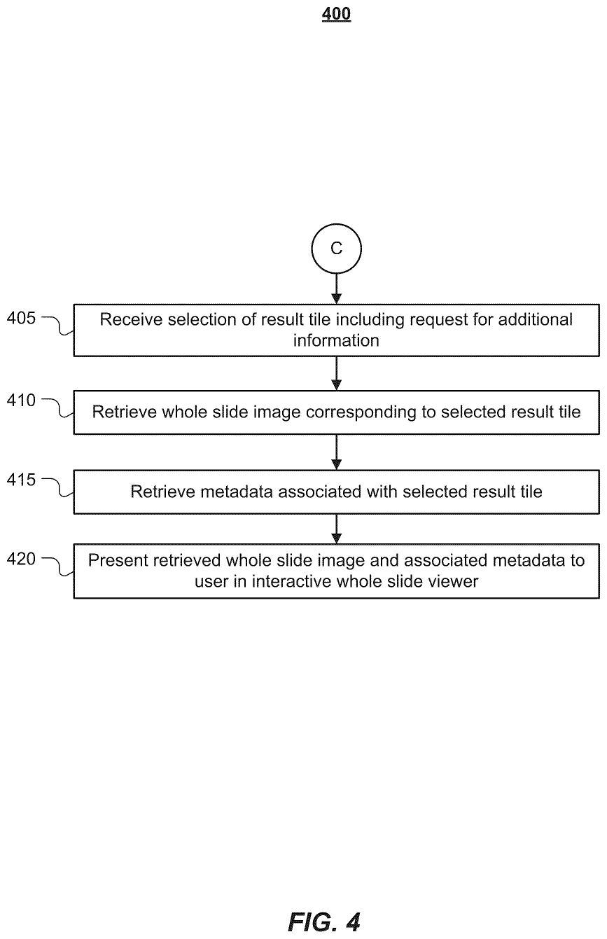

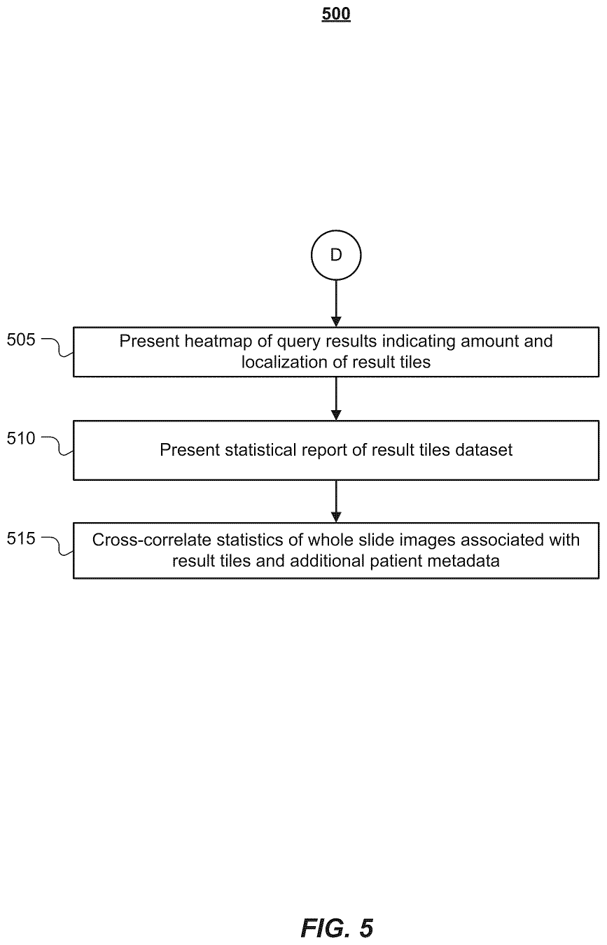

If, at step 145, the input is determined to be directed to the quality of the results (e.g., when the results include results for which the user is searching), the method proceeds to the method 200 for refinement by example selection in whole slide image search illustrated in FIG. 2, designated in FIG. 1 as element A. If, at step 150, the input is determined to be a request for the whole slide image search system to enter an object filter generation mode, the method proceeds to the method 300 for object filter generation in whole slide image search illustrated in FIG. 3, designated in FIG. 1 as element B. As discussed previously, the term object filter is used herein to refer to a filter generated based on a selection of one or more images or tiles. If, at step 155, the input is determined to be a selection of one or more of the result tiles, the method proceeds to the method 400 for whole slide output generation in whole slide image search illustrated in FIG. 4, designated in FIG. 1 as element C. If, at step 160, the input is determined to be a request for output reports, the method proceeds to the method 500 for dataset output generation in whole slide image search illustrated in FIG. 5, designated in FIG. 1 as element D. The method 100 may repeat in whole or in part multiple times, for example, the user may modify the query or query image slightly and request multiple sets of result tiles. Furthermore, the whole slide image search system may receive multiple forms of user input and may present more than one of the methods illustrated in FIGS. 2-5 as discussed herein.

FIG. 2 illustrates an example method 200 for refinement by example selection in whole slide image search. The method begins as a continuation from step 145 in FIG. 1. The method begins at step 205, where the whole slide image search system modifies the weighting associated with results which have been indicated by the user based on the user input relating to the quality of the results and particularly indicating whether the results positively or negatively reflect what results the user was expecting to be shown. For example, the whole slide image search system may collect the results that have been positively indicated and compute an average embedding for the positive results. The whole slide image search system may use this average embedding to modify the search query, such as by searching for embeddings most similar to the average embedding. Similarly, where the user has indicated negative results, the whole slide image search system may collect the results that have been negatively indicated and compute an average embedding for the negative results. The whole slide image search system may use this average embedding to modify the search query, such as by using the average embedding for the negative results as a contraindication of a similar result (e.g., as an indication of a dissimilar result). As another example, the whole slide image search system may increase the weighting associated with results that have been positively indicated and decrease the weighting associated with results that have been negatively weighted. These adjustments to the weightings can be back-propagated so that results similar to the positive results have a relatively higher score while results similar to the negative results have a relatively lower score. In particular embodiments, the determination for whether results are positive or negative can be enhanced with a ranking system that allows the user to provide more nuanced feedback on the results. The degree of the ranking system can be used to modify the weight attributed to positive or negative results.

At step 210, the whole slide image search system can receive a user input requesting for the whole slide image search system to re-run the search query based on the learned example. At step 215, the whole slide image search system can compare the embedding for the query image to the embeddings in the whole slide image dataset to determine the most similar set of embeddings using the average embedding for the positive results and average embedding of the negative results or the modified weighting using methods as described above. For example, the most similar set of embeddings may be determined to be the embeddings with the least distance to the average embedding for the positive results in the embeddings pace. As another example, the most similar set of embeddings may be the embeddings with the least distance to the query image in the feature embedding space, where the distance function used to determine proximity in the feature embedding space is based on the applied weightings.

At step 220, the whole slide image search system can identify result tiles based on the embeddings determined to be the most similar embeddings in the preceding step. At step 225, the whole slide image search system can present the identified result tiles. As in step 135 of the method 100, presenting the identified result tiles can involve presenting a subset of the result tiles in an interactive user interface on a client device. The interactive user interface can include a variety of interactive elements to enable the user to engage with further functions of the whole slide image search system. Although not illustrated, the whole slide image search system can monitor for user input relating to those additional functions.

FIG. 3 illustrates an example method 300 for object filter generation in whole slide image search. The method begins as a continuation from step 150 in FIG. 1. The method begins at step 305, where the whole slide image search system receives user input marking one or more of the presented result tiles from the query image. At step 310, the whole slide image search system receives user input of a weighting to associate with the marked result tiles. From steps 305 and 310, the whole slide image search system has a designation of example result tiles for the object filter and weightings to be attributed to the object filter. In particular embodiments, positive weightings are taken to be affirmative representations of the object filter, while negative ratings are taken to be examples of the absence of the object filter. Thus, the object filters can be generated dynamically, responsive to user search and feedback. In particular embodiments, the user can execute searches with the express intent to building out object filters for later use (e.g., using the described object filter saving, sharing, and loading features).

At step 315, the whole slide image search system generates an object filter based on the received user inputs. To generate the object filter, the whole slide image search system can train a new classifier from the received user inputs in order to learn to create a representation of the object filter, shown in the result tiles determined based on the received user inputs, in the embedded space. This representation can then be weighted and averaged with the embedding for the original query image to conduct a subsequent search. This results in finding embeddings that are similar to the augmented query the original query averaged with the learned object filter. As an example, the whole slide image search system can train a model (e.g., a linear support vector machine) using marked result tiles appended with the associated weightings. The object filter itself is represented by the support vector machine hyperplane which corresponds to the embedding for the object filter. A subsequent query can then efficiently use the selected object filter and associated weights to produce query results (e.g., result tiles) that are more similar to result tiles marked with a positive weighting and more dissimilar to result tiles marked with a negative weighting.

At step 320, the whole slide image search system receives a user input requesting for the whole slide image search system to re-run the search query using the newly generated object filter. At step 325, the whole slide image search system can compare the augmented query image (the embedding for the original query image averaged with the learned representation of the concept) to the embeddings in the whole slide image search dataset to determine the most similar embeddings to the augmented embedding for the query image to determine the most similar tiles to the augmented query image.

In some embodiments, at step 330, the whole slide image search system can apply a representation of the generated object filter to the identified nearest embeddings. For example, where the object filter is represented as an embedding generated from the support vector machine hyperplane, the object filter embedding can be compared against the nearest embeddings determined in the preceding step. The object filter can also be provided with a weight indicating how positive matches to the object filter (e.g., embeddings that are near the object filter in the feature embedding space) should be handled. If the weight is negative, then matches to the filter will be lowered in rank or excluded.

At step 335, the whole slide image search system identifies result tiles based on the determined nearest embeddings according to the techniques described above. At step 340, the whole slide image search system can present the identified result tiles. As in step 135 of the method 100, presenting the identified result tiles can involve presenting a subset of the result tiles in an interactive user interface on a client device. The interactive user interface can include a variety of interactive elements to enable the user to engage with further functions of the whole slide image search system. Although not illustrated, the whole slide image search system can monitor for user input relating to those additional functions.

At step 345, the whole slide image search system can receive a user input requesting the whole slide image search system to save the generated object filter for future use, e.g., by the user. As an example, the saved object filter can be indicated in step 115 of the method 100. As an example, the saved object filter can be saved to an account record associated with the user so that the user can access saved filters regardless of the user device used to access the whole slide image search system. In other embodiments, the saved object filter can be exported or downloaded to the user device so that is can be saved outside the account record.

At step 350, the whole slide image search system can receive a user input requesting the whole slide image search system to share the generated object filter with other users. For example, a network of users can be created, where each user has access to the whole slide image search system from their own user device. The network of users can be public, restricted to certain institutions or types of use for the whole slide image search system, or may otherwise be restricted. Object filters can be shared among the users, so that users can build on each other's successes. Furthermore, libraries of shared object filters can be developed and fine-tuned so that a new user does not need to build up common object filters from a true starting position. Instead, the user can access the library and easily have access to useful filters. Additionally, the user can receive object filters from other uses of the whole slide image search system.

FIG. 4 illustrates an example method 400 for whole slide output generation in whole slide image search. The method begins as a continuation from step 155 in FIG. 1. The method begins at step 405, where the whole slide image search system receives a selection of a result tile including a request for additional information about the result tile. For each, a user may double click a result tile, indicating that the user would like to see the result tile in context and/or to receive additional information about the result tile.

At step 410, the whole slide image search system retrieves the whole slide image corresponding to the selected result tile. As described herein, the result tile was segmented or excerpted from a whole slide image. The result tile may also have been stored with metadata to associate the result tile with the whole slide image. On receiving the user input, the whole slide image search system can retrieve the corresponding whole slide image from the appropriate database and prepare it for presentation to the user. For example, because whole slide images are large and data-intensive, the whole slide image search system can begin to stream the whole slide image or subsets thereof to the user device.

At step 415, the whole slide image search system retrieves metadata associated with the selected result tile and stored in appropriate databases of the whole slide image search system. In particular embodiments, the metadata can be specific to the individual tile. For example, the metadata can describe information relating to features shown in the individual tile, information about when and how the tile was generated, etc. The metadata can also provide information regarding the corresponding whole slide image. For example, the whole slide image may have been stored with information regarding the source of the whole slide image such as an identifier for the patient, the tissue being displayed, the sample from which the whole slide image was taken, the providing user (e.g., researcher), temporal features of the sample, scanner model, scan magnification, staining protocols, tissue thickness, and other related information. Furthermore, the whole slide image may have been stored in association with metadata regarding the patient, such as diagnoses, conditions, other irregularities of note, etc.

At step 420, the whole slide image search system presents the retrieved whole slide image and associated metadata to the user in an interactive whole slide viewer. The interactive whole slide viewer can show the selected result tile within the context of the whole slide image from which it was segmented. The interactive whole slide viewer can provide a variety of functions for the user to manipulate the whole slide image to better understand the selected result tile. The interactive whole slide viewer can provide zoom functionality to zoom in and out of the whole slide image, panning functionality to view different parts of the whole slide image, color modification (e.g., tint or hue adjustment, grayscale selection) to allow the user to focus on different aspects of the whole slide image, and other related functions. The whole slide image viewer can also present the corresponding metadata to the user. Therefore, the whole slide image viewer facilitates the user quickly evaluating and comparing against the result tile to the query image while also facilitating access to known end points, speeding up recognition and diagnostic capability.

From the whole slide image viewer, the user can also initiate other functions, such as initiate another search query by selecting a relevant portion of the whole slide image, can mark the result tile and/or whole slide image for future reference and retrieval, or return to the set of results from the original query.

FIG. 5 illustrates an example method 500 for dataset output generation in whole slide image search. The method begins as a continuation from step 160 in FIG. 1. Each of the steps in the method 500 can be performed responsive to user input. For example, the user may request the generation of an associated heatmap but may not request a statistical report on the result tile dataset in certain embodiments.

At step 505, where the whole slide image search system receives a request from the user to generate and present a heatmap of query results. In particular embodiments, the heatmap can be used to efficiently display information regarding locations in the whole slide image of interest. For example, the heatmap can provide information about the query image where the query image includes enough information to be useful for analytical purposes (e.g., were the query image was selected from a whole slide image). For example, the heatmap can include a display of the source of the query image that includes a comparison of the query image to the source itself. As another example, the heatmap can represent the similarity of each tile in a whole slide image to the query image (corresponding, for example, to the distance between an embedding generated for each tile in the whole slide image to the embedding generated for the query image). As another example, the heatmap can analyze the one or more whole slide images to display, for example, regions that are frequently requested by the user or other users, regions that are frequently accessed by users of the whole slide image search system, are frequently shown as top result tiles for other searches, etc. Furthermore, the heatmap can efficiently summarize the prevalence of particular histopathology phenotypes within the query image and whole slide image from which the query image was generated.

At step 510, the whole slide image search system receives a request from the user to generate and present a statistical report of the result tiles dataset, e.g., the result tiles returned and presented to the user in step 135 of method 100. For example, the whole slide image search system can determine and summarize the location of each result tile within its respective source whole slide image. The whole slide image search system can identify relationships between any result tiles, such as whether one or more of the result tiles originate from the same whole slide image or the same subject or providing user. The whole slide image search system can include summaries of classifications for the result tiles and the feature shown therein. The whole slide image search system can summarize temporal information for the result tiles, such as the age of the image and the age of the sample. The whole slide image search system can provide spatial statistics of the distribution of similar tiles in a given whole slide image, such as cluster locations, number of clusters, size of clusters, average cluster similarity (measured for example, based on average distance between embeddings corresponding to each tile of the cluster in the feature embedding space), average total similarity (measured, for example, based on an average value of the feature vector corresponding to each embedding generated based on the cluster or of one or more components of the feature vector), percentage of similar tiles, etc. The whole slide image search system can provide patient omics data, such as identified genomic or proteomic data or features for a patient, including, for example, strongest fold-change genes, and other omics biomarker expression. Although several example statistics have been provided, it will be appreciated that many suitable statistics regarding the result tiles dataset can be compiled and presented individually to the user or packaged before presentation.

At step 515, the whole slide image search system receives a request to generate and present cross-correlated statistics of the whole slide image associated with the result tiles. The cross-correlated report can further include information about the patients, the samples from whom were used to generate the whole slide images. For example, whereas the statistical report that may be produced in step 510 provides information about the result tiles specifically, the statistics gathered and presented in step 515 relate to the associated whole slide image and corresponding underlying patient information. The underlying patient information can include patient demographics, temporal relationships between the whole slide images (if they originate from the same patient), patient outcomes if known (e.g., overall survival, overall response, progression-free survival, recorded adverse events, etc.), patient diagnoses if material, the type of tissue depicted, the context for the sample such as the providing user or study, and other related information. Although several example statistics have been provided, it will be appreciated that many suitable statistics regarding the whole slide images associated with the result tiles can be compiled and presented individually to the user or packaged before presentation.

Particular embodiments may repeat one or more steps of the method of FIGS. 1-5, where appropriate. Although this disclosure describes and illustrates particular steps of the method of FIGS. 1-5 as occurring in a particular order, this disclosure contemplates any suitable steps of the method of FIGS. 1-5 occurring in any suitable order. Moreover, although this disclosure describes and illustrates example methods for whole slide image search including the particular steps of the methods of FIGS. 1-5, this disclosure contemplates any suitable method for whole slide image search including any suitable steps, which may include all, some, or none of the steps of the method of FIGS. 1-5, where appropriate. Furthermore, although this disclosure describes and illustrates particular components, devices, or systems carrying out particular steps of the method of FIGS. 1-5, this disclosure contemplates any suitable combination of any suitable components, devices, or systems carrying out any suitable steps of the method of FIG. 1-5.

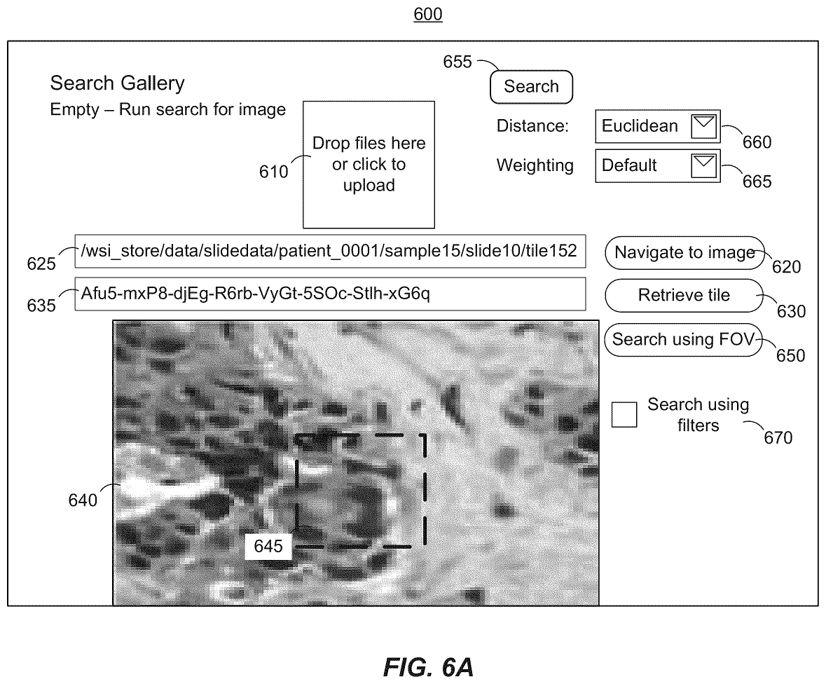

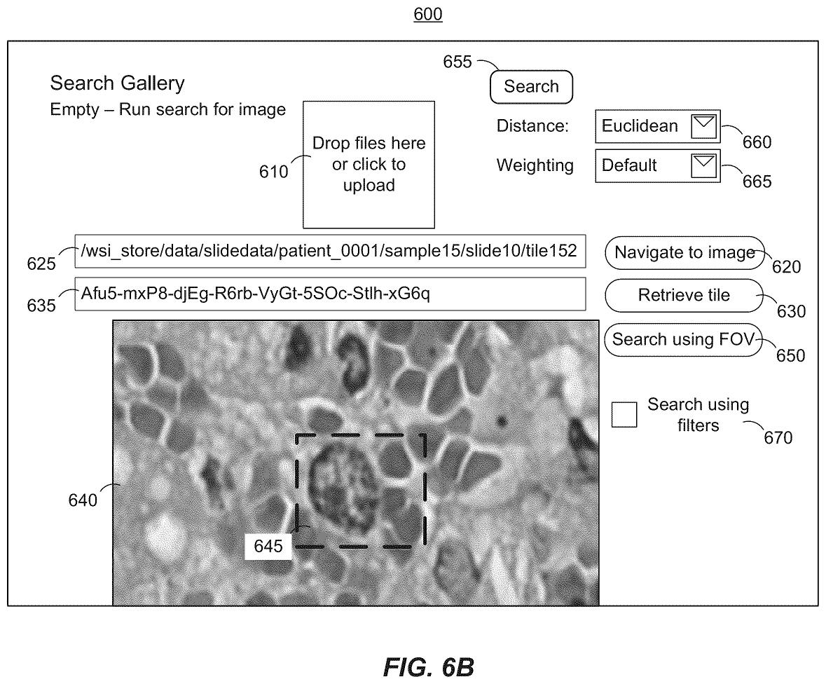

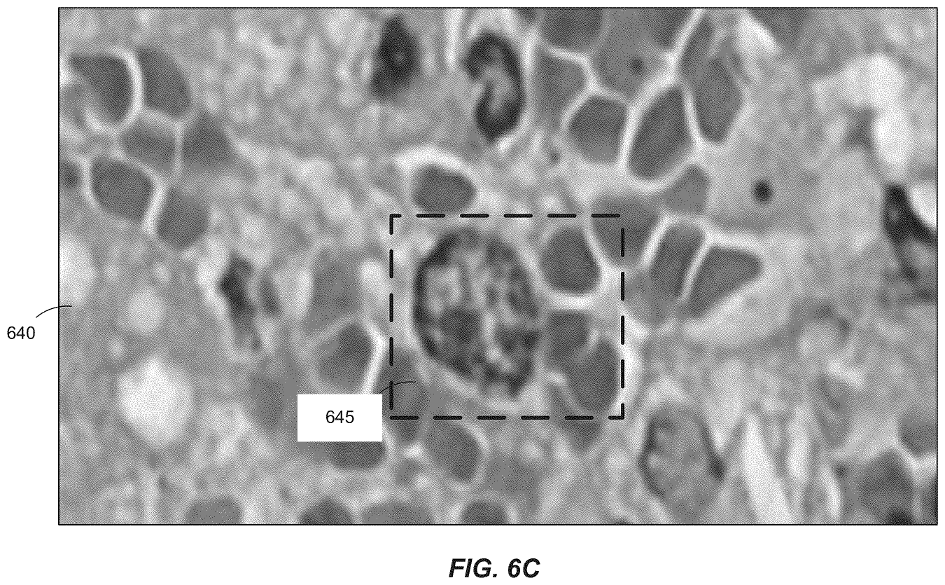

FIGS. 6A and 6B illustrate an example user interface 600 for providing a query image to the whole slide image search system as in the method 100 for whole slide image search. As shown, the user can provide user input in a variety of methods. The end result is for the user to provide and designate an image to be used as the basis for a search. User interface 600 includes a first interactive element 610 requesting a user to drag-and-drop an image file to be uploaded to the whole slide image search system for use as the query image. This may be helpful when a particular feature or phenotype is difficult to find in an available whole slide image (e.g., to search using a field of view, discussed below), but an example has been previously excerpted. Alternatively, the user may click or tap the first interactive element 610 to open a system browser to select the image for upload. The first user interface 610 can therefore be used to supply an image from a system external to the whole slide image search system (e.g., a user device) that is in communication with the whole slide image search system.

To facilitate a second method of specifying a query image, user interface 600 includes a second interactive element 620 and a text field 625 through which a user can specify the location or address (e.g., a file location or resource locator, which may be specified as a uniform resource locator) of a query image that is available to the whole slide image search system. For example, the whole slide image search system may be in communication with a database that stores patient or clinical data on behalf of users. The database may be accessible through the whole slide image search system, meaning that any device with access to the whole slide image search system has access to the database. The user can enter a file access path or resource locator corresponding to the query image into the text field 625. By interacting with the second interactive element 620, the user can instruct the whole slide image search system to retrieve the selected image (e.g., a whole slide image). Upon retrieval, the image can be displayed in the field 640, as shown in FIGS. 6A-6C. Field 640, as discussed below, is used to specify the specific area of the image to be searched.

To facilitate a third method of specifying a query image, user interface 600 includes a third interactive element 630 and text field 635 through which a user can provide a unique identifier for an image to be used as the query image. For example, the user can specify the unique identifier for a whole slide image that the user wishes to use as the query image. A database accessible by the whole slide image search system can be indexable by unique identifiers for the whole images and can retrieve the corresponding image upon selection of the third interactive element 630. Upon retrieval, the image can be displayed in the field 640.

Field 640 includes an image viewer, such as a whole slide image viewer where the selected image is a whole slide image. The image viewer can be operable to navigate the image (e.g., through zooming and panning) to facilitate the user reviewing the selected image. In some embodiments, the user may only use a portion of the displayed image as the query image. In such cases, the image viewer field 640 can include an adjustable field of view (FOV) selector 645. In particular embodiments, the FOV selector 645 can be directly and separately manipulated from the image viewer field 640. In some embodiments, the FOV selector 645 represents a predefined portion of the image (e.g., the center of the image) and manipulating the image viewer 640 also manipulates the FOV selector 645.

To submit the image as a query image and initiate the search, a user can select the appropriate search button 650 or 655. If the user has specified a region of a selected image to use for a query image using the FOV selector 645, the user can engage the search button 650 marked “Search using FOV.” This causes the whole slide image search system to interpret the selected FOV as a specification of a region of the whole slide image shown in the image viewer 640 and segment the region selected in the FOV from the presented image to be used as the query image. Alternatively, the user can select the search button 655 which will use the full image displayed in the image viewer 640 or otherwise provided to the whole slide image search system (e.g., using one of the input method discussed above).

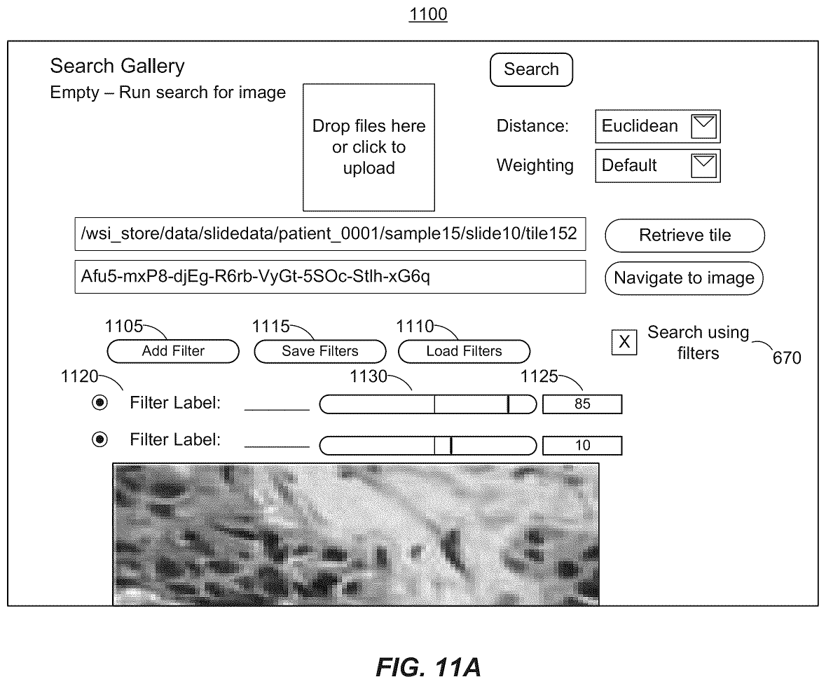

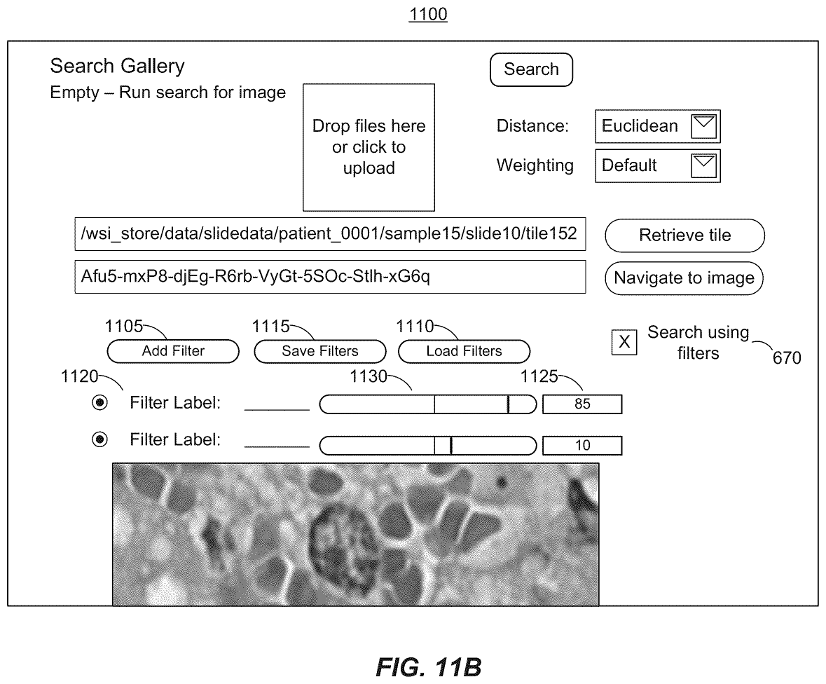

User interface 600 includes other inputs allowing the user to specify features of the search that will be conducted. A first input is the interactive element 660 through which a user can manually select the distance calculation that is used when comparing the embeddings stored in the whole slide image search dataset to the embedding generated from the query image. The interactive element 660 can provide a drop-down box showing the distance calculations currently made available by the whole slide image search system to the user. Another interactive element 665 allows the user to specify the weighting methodology used to rank and surface results to the user after the comparison is performed. The user can be provided an interface to customize the weighting methodology or other features of the result tile scoring methods that may not be captured in the embedding. A third interactive element 670 includes a toggle through which a user can indicate that they will specify an object filter to use when retrieving search results. When selected, the user interface 600 can be modified to provide for the method to specify the object filters to be used. This modified user interface 1100 is shown and described with respect to FIGS. 11A and 11B.

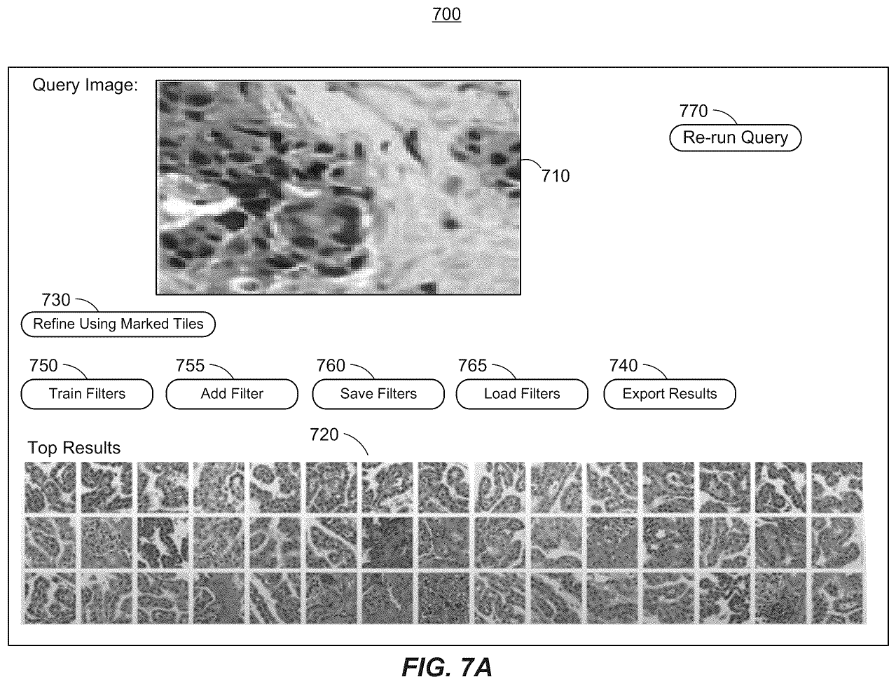

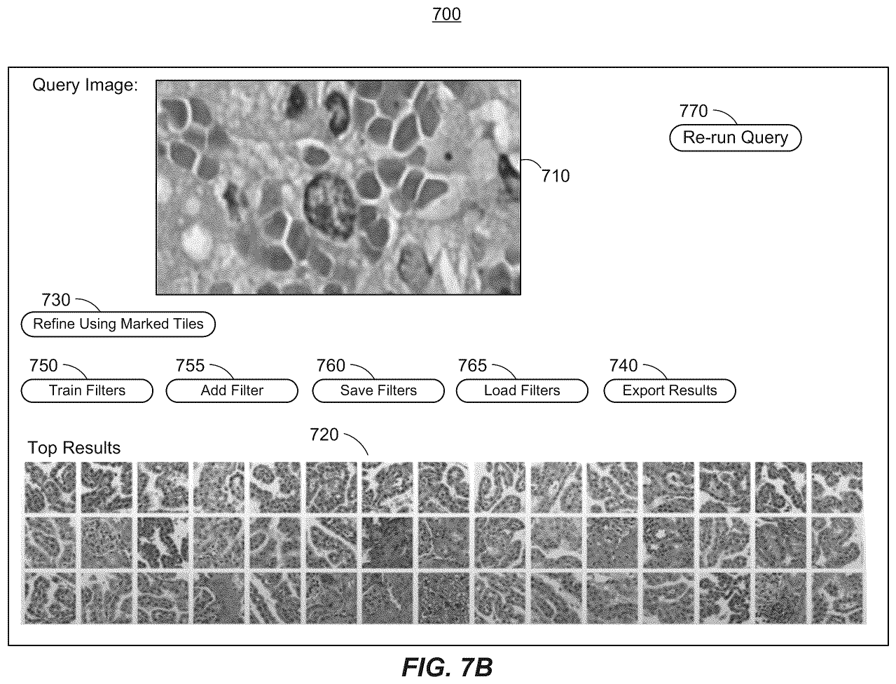

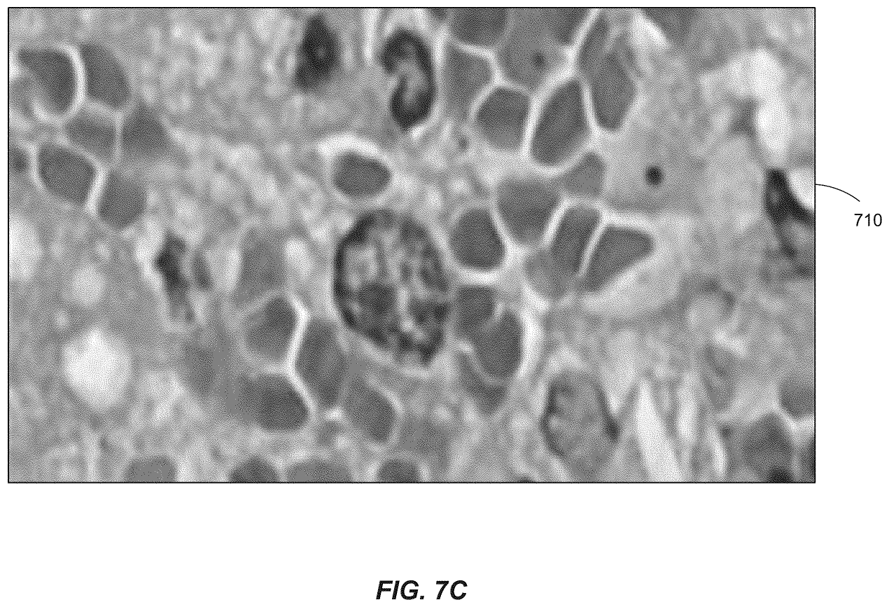

FIGS. 7A and 7B illustrate an example user interface 700 including tile results. The user interface 700 includes a display 710 that shows the query image used during the search for which results have been generated. An enlarged version of the display 170 is shown in FIG. 7C. The top result tiles 720 that have been identified by the whole slide image search system are shown toward the bottom the user interface 700 with a number of interactive elements included above the results that can be used by a user to take further action. In certain embodiments how the result tiles are shown to the user, such as the number of results, size of results, etc., can be customized to the user's preferences. Additionally, each of the result tiles can be interactive, allowing a user to review the tile result in further detail. When selected, interactive element 730 can initiate the refine by example mode, such as that described herein with respect to method 200. Interactive element 740 can be used to export the results for further analysis by the user. For example, the results can be packaged and downloaded to a user device along with relevant metadata and the corresponding whole slide image to allow for offline review. Additionally, the results can be exported to be shared with another user of the whole slide image search system.

Interactive elements 750, 755, 760, and 765 relate to the incorporation of object filters with the search results. When selected, interactive element 750 can initiate a train object filter mode used to generate object filters which can relate to the method 300 for object filter generation in whole slide image search described above. In particular embodiments, interactive elements 730 and 750 can cause the user interface 700 to transition to a new or modified interface configured to facilitate their stated purposes. Interactive element 755 allows a user to add a new object filter to use for refining the search results. Interactive element 760 allows a user to save an existing set of object filters for later recall, for example when the user has developed a set of object filters for use with specific types of query images and wishes to save the set of chosen filters for that purpose. Interactive element 765 allows the user to recall those chosen filters. When selected, interactive element 770 allows a user to re-run the query using the associated query image and incorporating any changes made by the user (e.g., using any of the described refinement techniques or using a new set of filters).

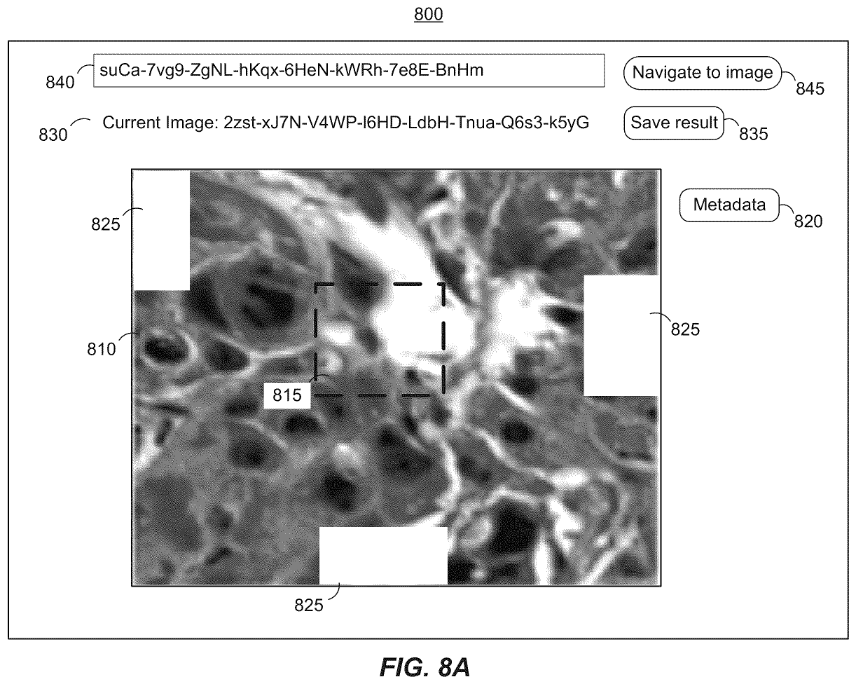

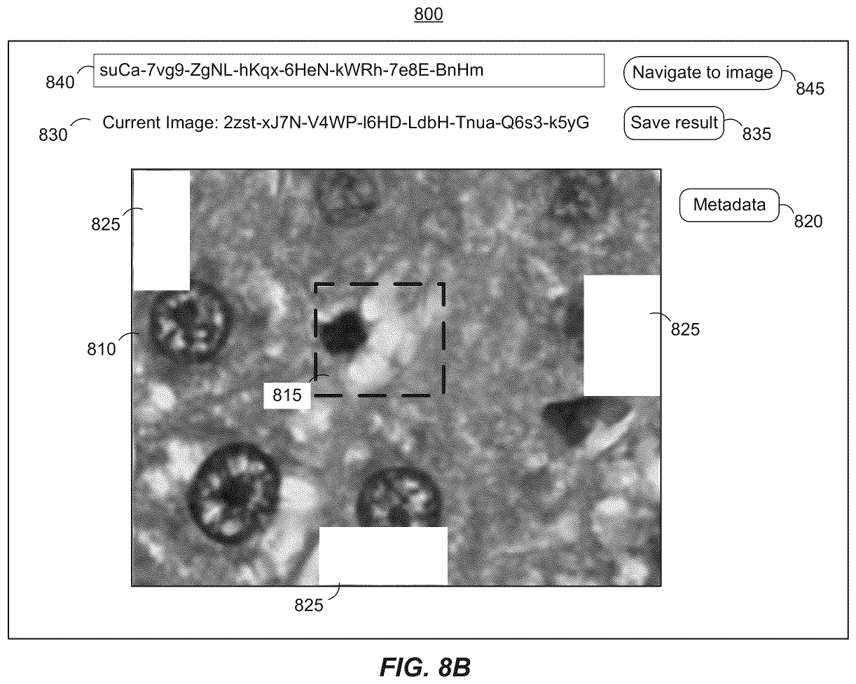

FIGS. 8A and 8B illustrate an example user interface 800 that includes a whole slide image result viewer 810. In certain embodiments, the user interface 800 can be accessed by selecting one of the result tiles presented in user interface 700. Additionally, user interface 800 can correlate to the method 400 for whole slide output generation in whole slide image search described herein. As described herein, the whole slide image viewer 810 can include controls to manipulate the whole slide image displayed therein, such as a zooming in or out of the image, panning across the image, and applying filters to facilitate easier review of the image. The whole slide image viewer 810 can further include an interface element 815 that denotes the position of the result tile that brought the user to this particular whole slide image. In this way, the whole slide image viewer 810 allows a user to understand the context of the result tile for use in understanding the whole slide image or for clinical and diagnostic purposes. Additionally, the user can select interactive element 820 to review metadata associated with the whole slide image. The metadata can include information such as data regarding the whole slide image (e.g., date of capture, size, other technical details), data regarding the sample from which the whole slide image was taken (e.g., type of tissue, date sample was taken), data regarding the patient from whom the sample was taken (e.g., patient status, diagnosis, survivability), and other relevant data that may be useful to the user in achieving their goals.

In certain embodiments, the whole slide image shown in the viewer 810 is displayed once all information has been loaded in. However, whole slide images can be very large, and it may take significant time to load the image into the viewer. Therefore, to improve the user experience and allow the user to view relevant portions of the whole slide image more quickly, the whole slide image may be loaded into the viewer in chunks. For example, the tiles or regions near the result tile 815 can be prioritized to be loaded before tiles that are further out. Shown in the whole slide image viewer 810 of this example, unloaded chunks 825 can be shown with placeholder data or merely be shown blanked out until the tile can be loaded into the viewer. Additionally, a progress bar can be provided to let the user track the progress of loading the tiles into whole slide image viewer. User interface 800 can also include a label 830 for the whole slide image shown in the viewer 810. This label can be used by the user to reference the whole slide image at a later time. Additionally or alternatively, the user can mark the image for saving, e.g., through interaction with the interactive element 835. The user interface 800 also serves as a whole slide image viewer for other whole slide images, including those not related to the result tile. Therefore, the user can use the text field 840 and interactive element 845 to identify and view another whole slide image by specifying, for example, a file path, resource locator, or unique identifier corresponding to the whole slide image. In particular embodiments, the user can execute another search from the viewer included in user interface 800.

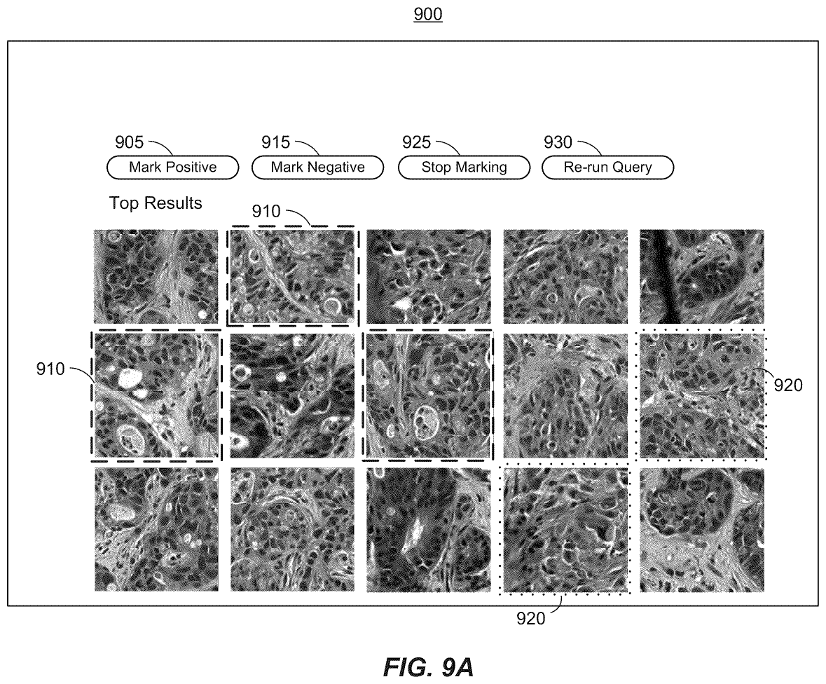

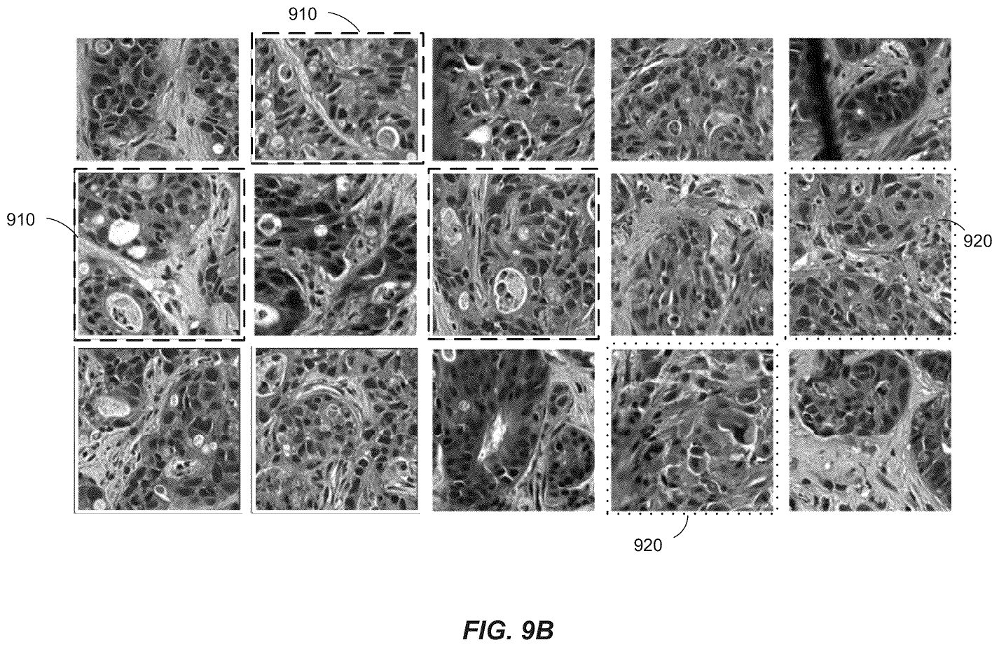

FIG. 9A illustrates an example user interface 900 that facilitates refinement by example selection in whole slide image search. As an example, user interface 900 can be reached by the user by interacting with the element 730 shown in FIGS. 7A and 7B, and may correspond to the method 200 for refinement by example selection in whole slide image search illustrated in FIG. 2 and discussed above. The user interface 900 includes the top result tiles identified for the query image. The user interface 900 also includes a number of interactive elements to allow the user to provide input to the whole slide image search system. For example, interactive element 905 allows the user to indicate that they are marking results as positive (indicating that the results are highly related to the query image). Interactive element 915 allows the user to indicate that they are marking results as negative (indicating that the results are not related to the query image). After selecting the interactive element 905, the user can select one or more of the result tiles. The display of the result tiles will then be modified to indicate to the user which result tiles they have marked as positive. In the user interface 900, the positively-marked tiles 910 are shown with a dashed line surrounding the tiles in FIGS. 9A and 9B. Other indicators can also be used, such as surrounding the positively-marked tiles in a certain color (e.g., green), shading or otherwise adjusting the hue of the tiles, etc.

To stop positively marking tiles, the user can select the interactive element 925 or, if they wish to proceed to negatively mark tiles, interactive element 915. Similar to positively marking tiles, after selecting interactive element 915, the user selects one or more of the result tiles. The display of the result tiles will then be modified to indicate to the user which result tiles they have marked as negative. In the user interface 900, the negatively-marked tiles 920 are shown with a dotted line surrounding the tiles in FIGS. 9A and 9B. Other indicators, distinct from the indicators for positively-marked tiles can be used, such as surrounding the negatively-marked tiles in a certain color (e.g., red), shading or otherwise adjusting the hue of the tiles, etc.

After the user has finished marking tiles, they can select interactive element 930 to re-run the query using the original query image, but results will be generated based on the refinement by example process described above with respect to method 200.

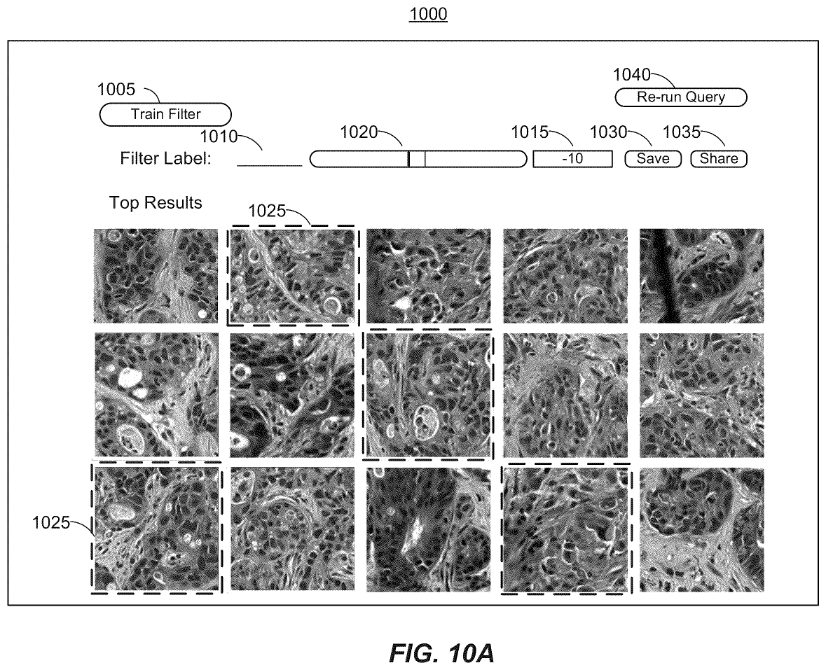

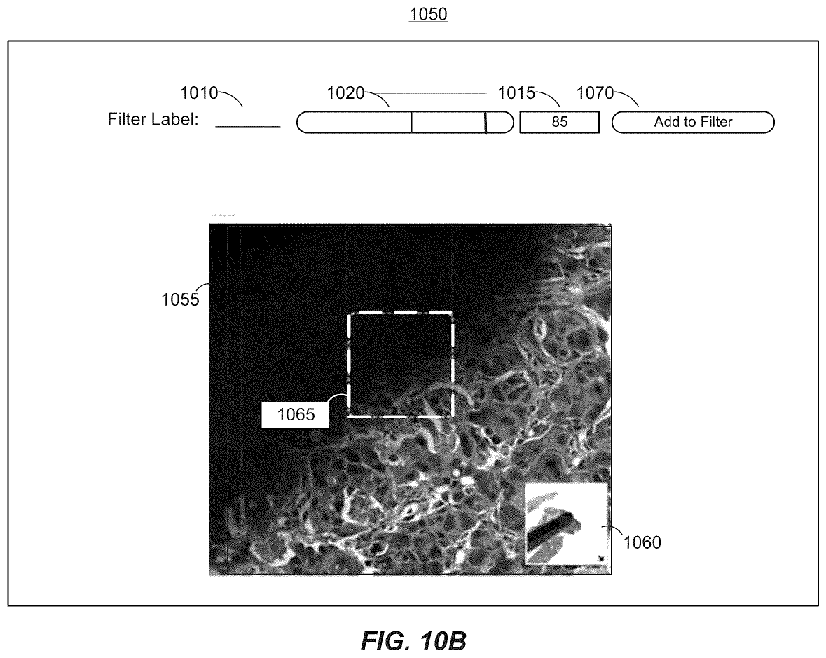

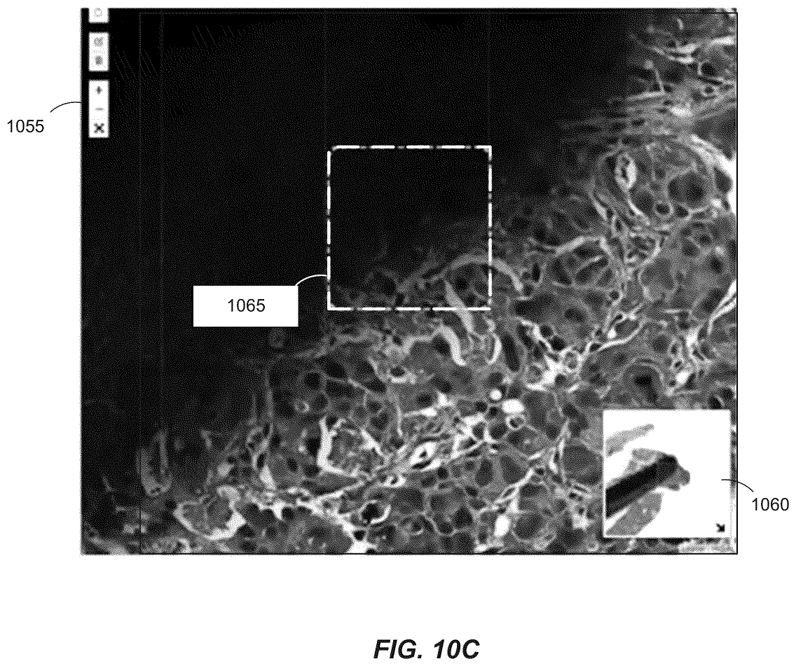

FIG. 10A illustrates an example user interface 1000 that includes object filter generation by marking result tiles. In particular embodiments, user interface 1000 can be access through selection of interactive element 750 in user interface 700. User interface 1000 includes a display field for the result tiles provided as a result of a query image. User interface 1000 also includes various interactive elements to facilitate generation and training of object filters. The user can create a label for the object filter using the text field 1010. This can allow the user to easily recall the purpose of the label, for example, to exclude aberrations in the result tiles caused by defects in the imaging process or to exclude certain tissue structures. Interactive elements 1015 and 1020 allow a user to modify a strength of the object filter or the strength of individual result tiles as examples of the object filter. Interactive element 1015 shows a numerical value (ranging from −100 to 100, although any suitable range will be acceptable) corresponding to the strength of the marked object filters to the filter. Interactive element 1020 shows the same information but using a sliding bar. The bar can also be color coded (e.g., with solid red corresponding to strongly negative, yellow indicating neutral, and strongly green corresponding to strongly positive) or include other easily identifiable visual information.

After selecting a weight and indication of positive or negative example, the user can select one or more of the result tiles. The display of the result tiles will then be modified to indicate to the user that they have selected the result tile as an example. The display can also be modified to indicate the relative weight attributed by the user to the particular result tile. In the user interface 1000, the marked tiles 1025 correspond to a −10 indication and are shown with dashed lines surrounding the tiles. The user can select interactive element 1005 to indicate that the appropriate tiles have been marked and indicate that the object filter is ready for generation. Depending on the complexity of the object filter, based for example on the number of result tiles that have been marked and the number of individual weighting indications that have been given, the user may have to wait some time before they are able to use the object filter. In such cases, the whole slide image search can indicate as much and continue to generate the object filter in the background while the user completes other tasks. The user can select the interactive element 1030 to save the generated object filter for future use and select the interactive element 1035 to share the generated object filter with one or more other users of the whole slide image search system. Additionally, the user can select interactive element 1040 to immediately re-run the search query using the provided query image and the generated object filter.

FIG. 10B illustrates an example user interface 1050 that includes object filter generation by identifying features of a whole slide image to mark. Using the user interface 1050, a user can select one or more features of a whole slide image (or just of an individual tile from a whole slide image) to indicate as an example for an object filter. Like the user interface 1000, the user interface 1050 includes an interactive element 1010 to apply a label to the object filter and interactive elements 1015 and 1020 to select a strength or weight of the example to the object filter. In addition to generating a new object filter, the user can also use the user interface 1050 to add an example to an existing object filter. User interface 1050 includes an image viewer 1055 that may function similarly to the whole slide image viewer 810 discussed above. The image viewer 1055 can also include a smaller window 1060 that shows the location of the focused image within a larger whole slide image. FIG. 10C illustrates an enlarged version of the image viewer 1055 and window 1060. The image viewer further includes a field 1065 that the user can manipulate to specifically select the portion of the image that is an example for the object filter. Once the user is satisfied with their selection, they can select the interactive element 1070 to add the selected region of the image to the object filter for training purposes.

FIGS. 11A and 11B show an example user interface 1100 for receiving query input for whole slide image search. In particular, FIGS. 11A and 11B show a user interface 1100 for initiating a whole slide image search using object filters. Many of the input features shown in FIGS. 11A and 11B are similar to those shown and discussed in user interface 600 of FIGS. 6A and 6B. For the sake of brevity, the description of these features will be omitted. In particular embodiments, when the user selects the interactive element 670 to enable search using object filters, the user interface 600 adjusts or advances to user interface 1100 to include the search by object filter features. An interactive element 1105 allows the user to add an object filter to the query. Selection of this interactive element can cause a selection interface to be provided, through which a user can select individual object filters to add. An interactive element 1110 allows the user to add a grouping of object filters or to load one or more object filters that may be stored outside of the whole slide image search system. As an example, the whole slide image search system can allow for users to export filters to be stored on local devices and can further allow for users to import those filters at a later time. The filters that have been added or loaded are displayed at interface element 1120. The object filters are identified by their label. The user can also alter the effect of the object filters as applied to a prospective search query. For example, using the weighting text input field 1125 or sliding scale 1130, the user can specify whether a given object filter should be treated positively (e.g., positive examples used to train the object filter are treated positively and negative examples used to train the object filter are treated negatively) or negatively (e.g., positive examples used to train the object filter are treated negatively and negative examples used to train the object filter are treated positively). Once the user is satisfied with the selection of object filters, the user can initiate the search or can save the object filters using the interactive element 1115.

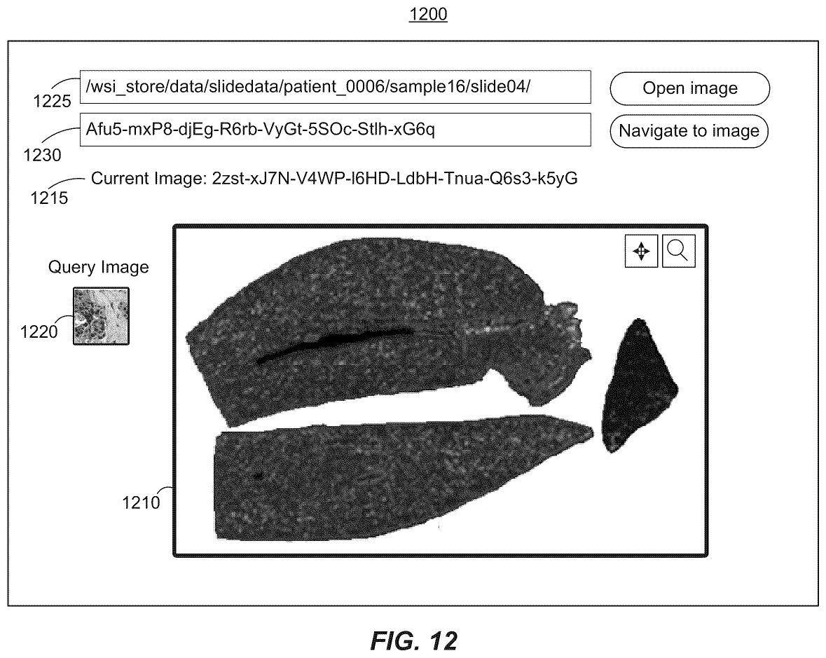

FIG. 12 shows an example user interface 1200 displaying output from the whole slide image search system. In particular, FIG. 12 shows a user interface 1200 comprising a heatmap 1210 of a particular whole slide image. In this example, the heatmap represents the similarity of each individual tile in the corresponding whole slide image to the query image (which may be measured, as described herein, based on the distance between an embedding generated for each tile of the whole slide image and an embedding generated for the query image in a feature embedding space), or another selected image such as an augmented query image described above. Lighter regions of the heatmap correspond to a higher degree of similarity while darker regions of the heatmap correspond to dissimilar tiles. The information represented in the heatmap can be used to localize areas in the depicted whole slide image that are similar to a specific query image. For example, a user may be looking for regions with a similar stroma structure, tissue anomalies, blood vessel structure, etc. The heatmap can further facilitate the user understanding, for example, the spatial distribution of these specific regions in a larger body, such as a tumor or other inspected tissue. The distribution of these objects in the tissue, shown in fuller context as in the whole slide image, can influence diagnosis, prognosis, or meta-analysis of the tissue sample (or patient from which the tissue sample was collected). The particular whole slide image is identified by the label display 1215. To assist the user's review of the heatmap, the user interface 1200 includes elements facilitating zooming into or out of the heatmap, panning across the heatmap, and may include other suitable elements for this purpose. To further assist review, the user interface 1200 includes a representation of the query image 1220. In particular embodiments, the user can switch between the heatmap display and a display of the whole slide image, allowing the user to directly compare the query image to regions of the whole slide image. Interactive element 1225 can receive a user input (e.g., specification of a file location, resource locator) to retrieve a locally or remotely stored image to convert to a heatmap comparing the tiles of the locally stored image to the query image and generate a heatmap similar to the heatmap 1210. Similarly, interactive element 1230 can receive a user input (e.g., unique identifier) to select a remotely stored image for the same purposes.

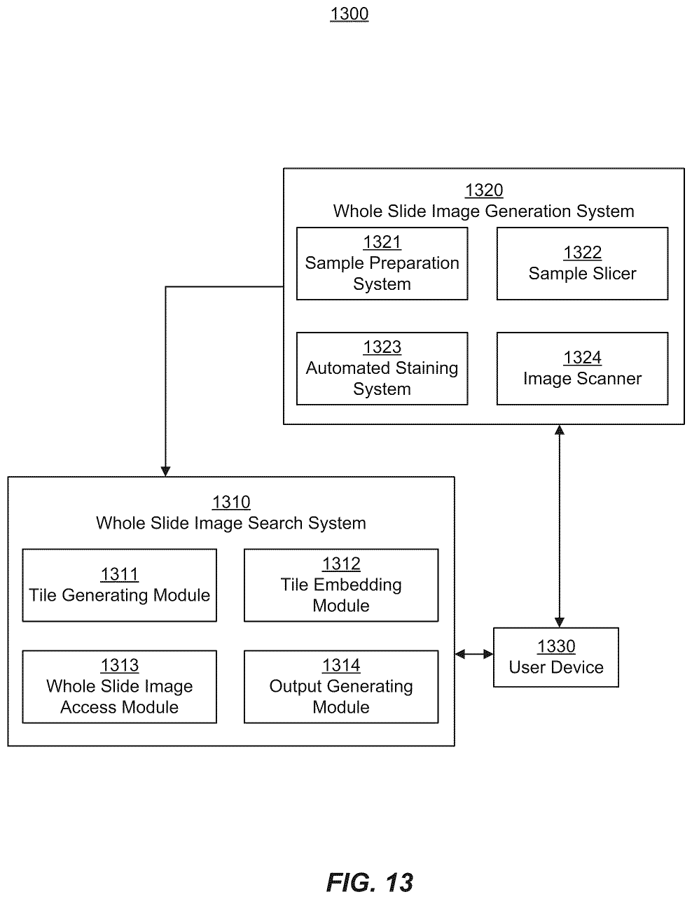

FIG. 13 illustrates a network 1300 of interacting computer systems that can be used, as described herein, for conducting whole slide image searching according to some embodiments of the present disclosure.

A whole slide image generation system 1320 can generate one or more whole slide images or other related digital pathology images, corresponding to a particular sample. For example, an image generated by whole slide image generation system 1320 can include a stained section of a biopsy sample. As another example, an image generated by whole slide image generation system 1320 can include a slide image (e.g., a blood film) of a liquid sample. As another example, an image generated by whole slide image generation system 1320 can include fluorescence microscopy such as a slide image depicting fluorescence in situ hybridization (FISH) after a fluorescent probe has been bound to a target DNA or RNA sequence.

Some types of samples (e.g., biopsies, solid samples and/or samples including tissue) can be processed by a sample preparation system 1321 to fix and/or embed the sample. Sample preparation system 1321 can facilitate infiltrating the sample with a fixating agent (e.g., liquid fixing agent, such as a formaldehyde solution) and/or embedding substance (e.g., a histological wax). For example, a sample fixation sub-system can fix a sample by exposing the sample to a fixating agent for at least a threshold amount of time (e.g., at least 3 hours, at least 6 hours, or at least 13 hours). A dehydration sub-system can dehydrate the sample (e.g., by exposing the fixed sample and/or a portion of the fixed sample to one or more ethanol solutions) and potentially clear the dehydrated sample using a clearing intermediate agent (e.g., that includes ethanol and a histological wax). A sample embedding sub-system can infiltrate the sample (e.g., one or more times for corresponding predefined time periods) with a heated (e.g., and thus liquid) histological wax. The histological wax can include a paraffin wax and potentially one or more resins (e.g., styrene or polyethylene). The sample and wax can then be cooled, and the wax-infiltrated sample can then be blocked out.

A sample slicer 1322 can receive the fixed and embedded sample and can produce a set of sections. Sample slicer 1322 can expose the fixed and embedded sample to cool or cold temperatures. Sample slicer 1322 can then cut the chilled sample (or a trimmed version thereof) to produce a set of sections. Each section can have a thickness that is (for example) less than 100 μm, less than 50 μm, less than 10 μm or less than 5 μm. Each section can have a thickness that is (for example) greater than 0.1 μm, greater than 1 μm, greater than 2 μm or greater than 4 μm. The cutting of the chilled sample can be performed in a warm water bath (e.g., at a temperature of at least 30° C., at least 35° C. or at least 40° C.).

An automated staining system 1323 can facilitate staining one or more of the sample sections by exposing each section to one or more staining agents. Each section can be exposed to a predefined volume of staining agent for a predefined period of time. In some instances, a single section is concurrently or sequentially exposed to multiple staining agents.

Each of one or more stained sections can be presented to an image scanner 1324, which can capture a digital image of the section. Image scanner 1324 can include a microscope camera. The image scanner 1324 can capture the digital image at multiple levels of magnification (e.g., using a 10× objective, 20× objective, 40× objective, etc.). Manipulation of the image can be used to capture a selected portion of the sample at the desired range of magnifications. Image scanner 1324 can further capture annotations and/or morphometrics identified by a human operator. In some instances, a section is returned to automated staining system 1323 after one or more images are captured, such that the section can be washed, exposed to one or more other stains and imaged again. When multiple stains are used, the stains can be selected to have different color profiles, such that a first region of an image corresponding to a first section portion that absorbed a large amount of a first stain can be distinguished from a second region of the image (or a different image) corresponding to a second section portion that absorbed a large amount of a second stain.

It will be appreciated that one or more components of whole slide image generation system 1320 can, in some instances, operate in connection with human operators. For example, human operators can move the sample across various sub-systems (e.g., of sample preparation system 1321 or of whole slide image generation system 1320) and/or initiate or terminate operation of one or more sub-systems, systems or components of whole slide image generation system 1320. As another example, part or all of one or more components of whole slide image generation system (e.g., one or more subsystems of the sample preparation system 1321) can be partly or entirely replaced with actions of a human operator.

Further, it will be appreciated that, while various described and depicted functions and components of whole slide image generation system 1320 pertain to processing of a solid and/or biopsy sample, other embodiments can relate to a liquid sample (e.g., a blood sample). For example, whole slide image generation system 1320 can receive a liquid-sample (e.g., blood or urine) slide that includes a base slide, smeared liquid sample and cover. Image scanner 1324 can then capture an image of the sample slide. Further embodiments of the whole slide image generation system 1320 can relate to capturing images of samples using advancing imaging techniques, such as FISH, described herein. For example, once a florescent probe has been introduced to a sample and allowed to bind to a target sequence appropriate imaging can be used to capture images of the sample for further analysis.

A given sample can be associated with one or more users (e.g., one or more physicians, laboratory technicians and/or medical providers) during processing and imaging. An associated user can include, by way of example and not of limitation, a person who ordered a test or biopsy that produced a sample being imaged, a person with permission to receive results of a test or biopsy, or a person who conducted analysis of the test or biopsy sample, among others. For example, a user can correspond to a physician, a pathologist, a clinician, or a subject. A user can use one or one user devices 1330 to submit one or more requests (e.g., that identify a subject) that a sample be processed by whole slide image generation system 1320 and that a resulting image be processed by a whole slide image search system 1310.

Whole slide image generation system 1320 can transmit an image produced by image scanner 1324 back to user device 1330. User device 1330 then communicates with the whole slide image search system 1310 to initiate automated processing of the image. In some instances, whole slide image generation system 1320 provides an image produced by image scanner 1324 to the whole slide image search system 1310 directly, e.g. at the direction of the user of a user device 1330. Although not illustrated, other intermediary devices (e.g., data stores of a server connected to the whole slide image generation system 1320 or whole slide image search system 1310) can also be used. Additionally, for the sake of simplicity only one whole slide image search system 1310, image generating system 1320, and user device 1330 is illustrated in the network 1300. This disclosure anticipates the use of one or more of each type of system and component thereof without necessarily deviating from the teachings of this disclosure.

The network 1300 and associated systems shown in FIG. 13 can be used in a variety of contexts where scanning and evaluation of digital pathology images, such as whole slide images, are an essential component of the work. As an example, the network 1300 can be associated with a clinical environment, where a user is evaluating the sample for possible diagnostic purposes. The user can review the image using the user device 1330 prior to providing the image to the whole slide image search system 1310. The user can provide additional information to the whole slide image search system 1310 that can be used to guide or direct the analysis of the image by the whole slide image search system 1310. For example, the user can provide a prospective diagnosis or preliminary assessment of features within the scan. The user can also provide additional context, such as the type of tissue being reviewed. As another example, the network 1300 can be associated with a laboratory environment were tissues are being examined, for example, to determine the efficacy or potential side effects of a drug. In this context, it can be commonplace for multiple types of tissues to be submitted for review to determine the effects on the whole body of said drug. This can present a particular challenge to human scan reviewers, who may need to determine the various contexts of the images, which can be highly dependent on the type of tissue being imaged. These contexts can optionally be provided to the whole slide image search system 1310.

Whole slide image search system 1310 can process digital pathology images, including whole slide images, to classify the digital pathology images and generate annotations for the digital pathology images and related output. A tile generating module 1311 can define a set of tiles for each digital pathology image. To define the set of tiles, the tile generating module 1311 can segment the digital pathology image into the set of tiles. As embodied herein, the tiles can be non-overlapping (e.g., each tile includes pixels of the image not included in any other tile) or overlapping (e.g., each tile includes some portion of pixels of the image that are included in at least one other tile). Features such as whether or not tiles overlap, in addition to the size of each tile and the stride of the window (e.g., the image distance or pixels between a tile and a subsequent tile) can increase or decrease the data set for analysis, with more tiles (e.g., through overlapping or smaller tiles) increasing the potential resolution of eventual output and visualizations. In some instances, tile generating module 1311 defines a set of tiles for an image where each tile is of a predefined size and/or an offset between tiles is predefined. Furthermore, the tile generating module 1311 can create multiple sets of tiles of varying size, overlap, step size, etc., for each image. In some embodiments, the digital pathology image itself can contain tile overlap, which may result from the imaging technique. Even segmentation without tile overlapping can be a preferable solution to balance tile processing requirements and avoid influencing the embedding generation and weighting value generation discussed herein. A tile size or tile offset can be determined, for example, by calculating one or more performance metrics (e.g., precision, recall, accuracy, and/or error) for each size/offset and by selecting a tile size and/or offset associated with one or more performance metrics above a predetermined threshold and/or associated with one or more optimal (e.g., high precision, highest recall, highest accuracy, and/or lowest error) performance metric(s).

The tile generating module 1311 may further define a tile size depending on the type of abnormality being detected. For example, the tile generating module 1311 can be configured with awareness of the type(s) of tissue abnormalities that the whole slide image search system 1310 will be searching for and can customize the tile size according to the tissue abnormalities to optimize detection. For example, the tile generating module 1311 can determine that, when the tissue abnormalities include searching for inflammation or necrosis in lung tissue, the tile size should be reduced to increase the scanning rate, while when the tissue abnormalities include abnormalities with Kupffer cells in liver tissues, the tile size should be increased to increase the opportunities for the whole slide image search system 1310 to analyze the Kupffer cells holistically. In some instances, tile generating module 1311 defines a set of tiles where a number of tiles in the set, size of the tiles of the set, resolution of the tiles for the set, or other related properties, for each image is defined and held constant for each of one or more images.

As embodied herein, the tile generating module 1311 can further define the set of tiles for each digital pathology image along one or more color channels or color combinations. As an example, digital pathology images received by whole slide image search system 1310 can include large-format multi-color channel images having pixel color values for each pixel of the image specified for one of several color channels. Example color specifications or color spaces that can be used include the RGB, CMYK, HSL, HSV, or HSB color specifications. The set of tiles can be defined based on segmenting the color channels and/or generating a brightness map or greyscale equivalent of each tile. For example, for each segment of an image, the tile generating module 1311 can provide a red tile, blue tile, green tile, and/or brightness tile, or the equivalent for the color specification used. As explained herein, segmenting the digital pathology images based on segments of the image and/or color values of the segments can improve the accuracy and recognition rates of the networks used to generating embeddings for the tiles and image and to produce classifications of the image. Additionally, the whole slide image search system 1310, e.g., using tile generating module 1311, can convert between color specifications and/or prepare copies of the tiles using multiple color specifications. Color specification conversions can be selected based on a desired type of image augmentation (e.g., accentuating or boosting particular color channels, saturation levels, brightness levels, etc.). Color specification conversions can also be selected to improve compatibility between whole slide image generation systems 1320 and the whole slide image search system 1310. For example, a particular image scanning component can provide output in the HSL color specification and the models used in the whole slide image search system 1310, as described herein, can be trained using RGB images. Converting the tiles to the compatible color specification can ensure the tiles can still be analyzed. Additionally, the whole slide image search system can up-sample or down-sample images that are provided in particular color depth (e.g., 8-bit, 16-bit, etc.) to be usable by the whole slide image search system. Furthermore, the whole slide image search system 1310 can cause tiles to be converted according to the type of image that has been captured (e.g., fluorescent images may include greater detail on color intensity or a wider range of colors).

As described herein, a tile embedding module 1312 can generate an embedding for each tile in a corresponding feature embedding space. The embedding can be represented by the whole slide image search system 1310 as a feature vector for the tile. The tile embedding module 1312 can use a neural network (e.g., a convolutional neural network) to generate a feature vector that represents each tile of the image. In particular embodiments, the tile embedding neural network can be based on the ResNet image network trained on a dataset based on natural (e.g., non-medical) images, such as the ImageNet dataset. By using a non-specialized tile embedding network, the tile embedding module 1312 can leverage known advances in efficiently processing images to generating embeddings. Furthermore, using a natural image dataset allows the embedding neural network to learn to discern differences between tile segments on a holistic level.

In other embodiments, the tile embedding network used by the tile embedding module 1312 can be an embedding network customized to handle large numbers of tiles of large format images, such as digital pathology whole slide images. Additionally, the tile embedding network used by the tile embedding module 1312 can be trained using a custom dataset. For example, the tile embedding network can be trained using a variety of samples of whole slide images or even trained using samples relevant to the subject matter for which the embedding network will be generating embeddings (e.g., scans of particular tissue types). Training the tile embedding network using specialized or customized sets of images can allow the tile embedding network to identify finer differences between tiles which can result in more detailed and accurate distances between tiles in the feature embedding space at the cost of additional time to acquire the images and the computational and economic cost of training multiple tile generating networks for use by the tile embedding module 1312. The tile embedding module 1312 can select from a library of tile embedding networks based on the type of images being processed by the whole slide image search system 1310.

As described herein, tile embeddings can be generated from a deep learning neural network using visual features of the tiles. Tile embeddings can be further generated from contextual information associated with the tiles or from the content shown in the tile. For example, a tile embedding can include one or more features that indicate and/or correspond to a size of depicted objects (e.g., sizes of depicted cells or aberrations) and/or density of depicted objects (e.g., a density of depicted cells or aberrations). Size and density can be measured absolutely (e.g., width expressed in pixels or converted from pixels to nanometers) or relative to other tiles from the same digital pathology image, from a class of digital pathology images (e.g., produced using similar techniques or by a single whole slide image generation system or scanner), or from a related family of digital pathology images. Furthermore, tiles can be classified prior to the tile embedding module 1312 generating embeddings for the tiles such that the tile embedding module 1312 considers the classification when preparing the embeddings.

For consistency, the tile embedding module 1312 produces embeddings of a predefined size (e.g., vectors of 512 elements, vectors of 2048 bytes, etc.). The tile embedding module 1312 can produce embeddings of various and arbitrary sizes. The time embedding module 1312 can adjust the sizes of the embeddings based on user direction or can be selected, for example, to optimize computation efficiency, accuracy, or other parameters. In particular embodiments, the embedding size can be based on the limitations or specifications of the deep learning neural network that generated the embeddings. Larger embedding sizes can be used to increase the amount of information captured in the embedding and improve the quality and accuracy of results, while smaller embedding sizes can be used to improve computational efficiency.

A whole slide image access module 1313 can manage requests to access whole slide images from other modules of the whole slide image search system 1310 and the user device 1330. For example, the whole slide image access module 1313 receive requests to identify a whole slide image based on a particular tile, an identifier for the tile, or an identifier for the whole slide image. The whole slide image access module 1313 can perform tasks of confirming that the whole slide image is available to the user requesting, identifying the appropriate databases from which to retrieve the requested whole slide image, and retrieving any additional metadata that may be of interest to the requesting user or module. Additionally, the whole slide image access module 1313 can handle efficiently streaming the appropriate data to the requesting device. As described herein, whole slide images may be provided to user devices in chunks, based on the likelihood that a user will wish to see the portion of the whole slide image. The whole slide image access module 1313 can determine which regions of the whole slide image to provide and determine how best to provide them. Furthermore, the whole slide image access module 1313 can be empowered within the whole slide image search system 1310 to ensure that no individual component locks up or otherwise misuses a database or whole slide image to the detriment of other components or users.

An output generating module 1314 of the whole slide image search system 1310 can generate output corresponding to result tile and result whole slide image datasets based on user request. As described herein, the output can include a variety of visualizations, interactive graphics, and reports based upon the type of request and the type of data that is available. In many embodiments, the output will be provided to the user device 1330 for display, but in certain embodiments the output can be accessed directly from the whole slide image search system 1310. The output will be based on existence of and access to the appropriate data, so the output generating module will be empowered to access necessarily metadata and anonymized patient information as needed. As with the other modules of the whole slide image search system 1310, the output generating module 1314 can be updated and improved in a modular fashion, so that new output features can be provided to users without requiring significant downtime.

The general techniques described herein can be integrated into a variety of tools and use cases. For example, as described, a user (e.g., pathology or clinician) can access a user device 1330 that is in communication with the whole slide image search system 1310 and provide a query image for analysis. The whole slide image search system 1310, or the connection to the whole slide image search system can be provided as a standalone software tool or package that searches for corresponding matches, identifies similar features, and generates appropriate output for the user upon request. As a standalone tool or plug-in that can be purchased or licensed on a streamlined basis, the tool can be used to augment the capabilities of a research or clinical lab. Additionally, the tool can be integrated into the services made available to the customer of whole slide image generation systems. For example, the tool can be provided as a unified workflow, where a user who conducts or requests a whole slide image to be created automatically receives a report of noteworthy features within the image and/or similar whole slide images that have been previously indexed. Therefore, in addition to improving whole slide image analysis, the techniques can be integrated into existing systems to provide additional features not previously considered or possible.

Moreover, the whole slide image search system 1310 can be trained and customized for use in particular settings. For example, the whole slide image search system 1310 can be specifically trained for use in providing insights relating to specific types of tissue (e.g., lung, heart, blood, liver, etc.). As another example, the whole slide image search system 1310 can be trained to assist with safety assessment, for example in determining levels or degrees of toxicity associated with drugs or other potential therapeutic treatments. Once trained for use in a specific subject matter or use case, the whole slide image search system 1310 is not necessarily limited to that use case. Training may be performed in a particular context, e.g., toxicity assessment, due to a relatively larger set of at least partially labeled or annotated images.

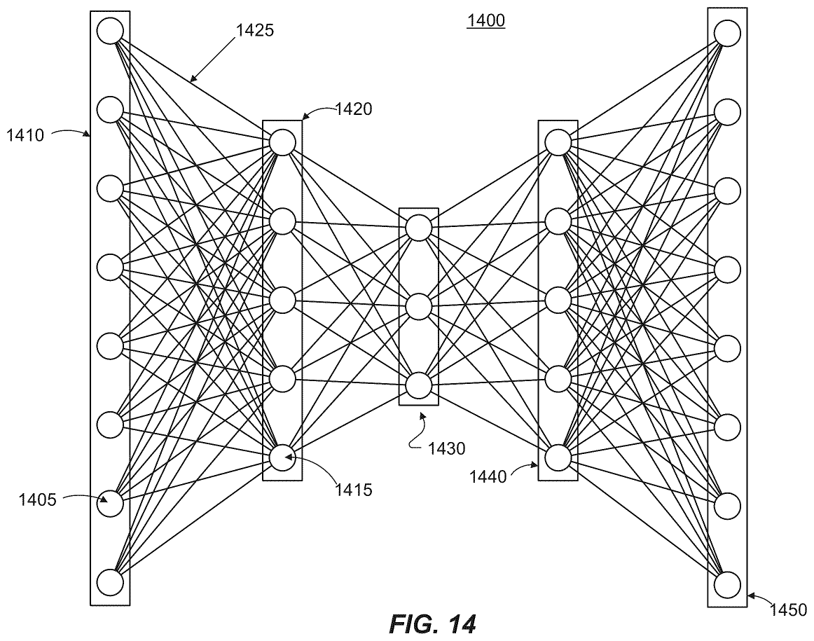

As described herein, the tile embedding network can be an artificial neural network (“ANN”) designed and trained for a specific function. FIG. 14 illustrates an example ANN 1400. An ANN can refer to a computational model comprising one or more nodes. An example ANN 1400 includes an input layer 1410, hidden layers 1420, 1430, 1440, and an output layer 1450. Each layer of the ANN 1400 can include one or more nodes, such as a node 1405 or a node 1415. In particular embodiments, one or more nodes of an ANN can be connected to another node of the ANN. In a fully-connected ANN, each node of an ANN is connected to each node of the preceding and/or subsequent layers of the ANN. As an example and not by way of limitation, each node of the input layer 1410 can be connected to each node of the hidden layer 1420, each node of the hidden layer 1420 can be connected to each node of hidden layer 1430, and so on. In particular embodiments, one or more nodes is a bias node, which can be a node that is not connected to and does not receive input from any node in a previous layer. Although FIG. 14 depicts a particular ANN 1400 with a particular number of layers, a particular number of nodes, and particular connections between nodes, this disclosure contemplates any suitable ANN with any suitable number of layers, any suitable number of nodes, and any suitable connections between nodes. As an example, FIG. 14 depicts a connection between each node of the input layer 1410 and each node of the hidden layer 1420, although in particular embodiments, one or more nodes of the input layer 1410 is not connected to one or more nodes of the hidden layer 1420 and the same applies for the remaining nodes and layers of the ANN 1400.

ANNs used in particular embodiments can be a feedforward ANN with no cycles or loops and where communication between nodes flows in one direction beginning with the input layer and proceeding to successive layers. As an example, the input to each node of the hidden layer 1420 can include the output of one or more nodes of the input layer 1410. Similarly, the input to each node of the output layer 1450 can include the output of nodes of the hidden layer 1440. ANNs used in particular embodiments can be deep neural networks having least two hidden layers. ANNs used in particular embodiments can be deep residual networks, a feedforward ANN including hidden layers organized into residual blocks. The input into each residual block after the first residual block can be a function of the output of the previous residual block and the input of the previous residual block. As an example and not by way of limitation, the input into residual block N can be represented as F(x)+x, where F(x) is the output of residual block N−1, and x is the input into residual block N−1. Although this disclosure describes a particular ANN, this disclosure contemplates any suitable ANN.

In particular embodiments, each node of an ANN can include an activation function. The activation function of a node defines or describes the output of the node for a given input. In particular embodiments, the input to a node can be a singular input or can include a set of inputs. Example activation functions can include an identity function, a binary step function, a logistic function, or any other suitable function. Example activation functions for a node k can include the sigmoid function

$${F}_{k} ( {s}_{k} ) = \frac{1}{1 + {e}^{- {s}_{k}}} ,$$

the hyperbolic tangent function

$${F}_{k} ( {s}_{k} ) = \frac{{e}^{{s}_{k}} - {e}^{- {s}_{k}}}{{e}^{{s}_{k}} + {e}^{- {s}_{k}}} ,$$

the rectifier Fₖ(sₖ)=max(0,sₖ), or any other suitable function Fₖ(sₖ), where sₖ is the input to node k.

The input of an activation function corresponding to a node can be weighted. Each node can generate output using a corresponding activation function based on weighted inputs. As embodied herein, each connection between nodes can be associated with a weight. For example, a connection 1425 between the node 1405 and the node 1415 can have a weighting coefficient of 0.4, which indicates that the input of node 1415 is 0.4 (the weighting coefficient) multiplied by the output of the node 1405. More generally, the output yₖ of node k can be yₖ=Fₖ(sₖ), where Fₖ is the activation function corresponding to node k, sₖ=Σⱼ(wⱼₖxⱼ) is the input to node k, xⱼ is the output of a node j connected to node k, and wⱼₖ is the weighting coefficient between node j and node k. As embodied herein, the input to nodes of the input layer 1410 can be based on a vector representing an object, also referred to as a vector representation of the object, an embedding of the object in a corresponding embedding space, or other suitable input. Although this disclosure describes particular inputs to and outputs of nodes, this disclosure contemplates any suitable inputs to and outputs of nodes in an ANN. Moreover, although this disclosure describes particular connections and weights between nodes, this disclosure contemplates any suitable connections and weights between nodes.

In particular embodiments, an ANN 1400 can be trained using training data. As an example and not by way of limitation, training data can include inputs to the ANN 1400 and an expected output, such as a ground truth value corresponding to the input. For example, training data can include one or more vectors representing a training object and an expected label for the training object. Training typically occurs with multiple training objects simultaneously or in succession. Training an ANN can include modifying the weights associated with the connections between nodes of the ANN by optimizing an objective function. As an example and not by way of limitation, a training method can be used to backpropagate an error value. The error value can be measured as a distance between each vector representing a training object, for example, using a cost function that minimizes error or a value derived from the error, such as a sum-of-squares error. Example training methods include, but are not limited to the conjugate gradient method, the gradient descent method, the stochastic gradient descent, etc. In particular embodiments, an ANN can be trained using a dropout technique in which one or more nodes are temporarily omitted while training such that they receive no input or produce no output. For each training object, one or more nodes of the ANN have a probability of being omitted. The nodes that are omitted for a particular training object can differ from nodes omitted for other training objects. Although this disclosure describes training an ANN in a particular manner, this disclosure contemplates training an ANN in any suitable manner.

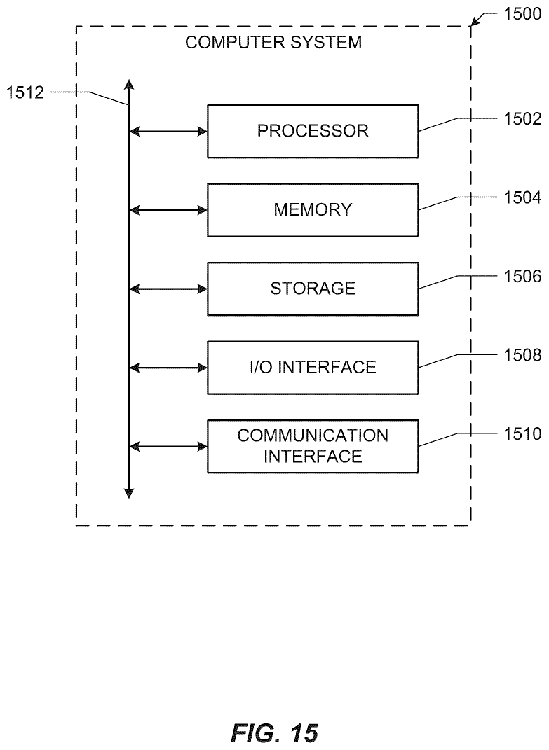

FIG. 15 illustrates an example computer system 1500. In particular embodiments, one or more computer systems 1500 perform one or more steps of one or more methods described or illustrated herein. In particular embodiments, one or more computer systems 1500 provide functionality described or illustrated herein. In particular embodiments, software running on one or more computer systems 1500 performs one or more steps of one or more methods described or illustrated herein or provides functionality described or illustrated herein. Particular embodiments include one or more portions of one or more computer systems 1500. Herein, reference to a computer system may encompass a computing device, and vice versa, where appropriate. Moreover, reference to a computer system may encompass one or more computer systems, where appropriate.

This disclosure contemplates any suitable number of computer systems 1500. This disclosure contemplates computer system 1500 taking any suitable physical form. As example and not by way of limitation, computer system 1500 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 1500 may include one or more computer systems 1500; be unitary or distributed; span multiple locations; span multiple machines; span multiple data centers; or reside in a cloud, which may include one or more cloud components in one or more networks. Where appropriate, one or more computer systems 1500 may perform without substantial spatial or temporal limitation one or more steps of one or more methods described or illustrated herein. As an example and not by way of limitation, one or more computer systems 1500 may perform in real time or in batch mode one or more steps of one or more methods described or illustrated herein. One or more computer systems 1500 may perform at different times or at different locations one or more steps of one or more methods described or illustrated herein, where appropriate.

In particular embodiments, computer system 1500 includes a processor 1502, memory 1504, storage 1506, an input/output (I/O) interface 1508, a communication interface 1510, and a bus 1512. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1502 includes hardware for executing instructions, such as those making up a computer program. As an example and not by way of limitation, to execute instructions, processor 1502 may retrieve (or fetch) the instructions from an internal register, an internal cache, memory 1504, or storage 1506; decode and execute them; and then write one or more results to an internal register, an internal cache, memory 1504, or storage 1506. In particular embodiments, processor 1502 may include one or more internal caches for data, instructions, or addresses. This disclosure contemplates processor 1502 including any suitable number of any suitable internal caches, where appropriate. As an example and not by way of limitation, processor 1502 may include one or more instruction caches, one or more data caches, and one or more translation lookaside buffers (TLBs). Instructions in the instruction caches may be copies of instructions in memory 1504 or storage 1506, and the instruction caches may speed up retrieval of those instructions by processor 1502. Data in the data caches may be copies of data in memory 1504 or storage 1506 for instructions executing at processor 1502 to operate on; the results of previous instructions executed at processor 1502 for access by subsequent instructions executing at processor 1502 or for writing to memory 1504 or storage 1506; or other suitable data. The data caches may speed up read or write operations by processor 1502. The TLBs may speed up virtual-address translation for processor 1502. In particular embodiments, processor 1502 may include one or more internal registers for data, instructions, or addresses. This disclosure contemplates processor 1502 including any suitable number of any suitable internal registers, where appropriate. Where appropriate, processor 1502 may include one or more arithmetic logic units (ALUs); be a multi-core processor; or include one or more processors 1502. Although this disclosure describes and illustrates a particular processor, this disclosure contemplates any suitable processor.

In particular embodiments, memory 1504 includes main memory for storing instructions for processor 1502 to execute or data for processor 1502 to operate on. As an example and not by way of limitation, computer system 1500 may load instructions from storage 1506 or another source (such as, for example, another computer system 1500) to memory 1504. Processor 1502 may then load the instructions from memory 1504 to an internal register or internal cache. To execute the instructions, processor 1502 may retrieve the instructions from the internal register or internal cache and decode them. During or after execution of the instructions, processor 1502 may write one or more results (which may be intermediate or final results) to the internal register or internal cache. Processor 1502 may then write one or more of those results to memory 1504. In particular embodiments, processor 1502 executes only instructions in one or more internal registers or internal caches or in memory 1504 (as opposed to storage 1506 or elsewhere) and operates only on data in one or more internal registers or internal caches or in memory 1504 (as opposed to storage 1506 or elsewhere). One or more memory buses (which may each include an address bus and a data bus) may couple processor 1502 to memory 1504. Bus 1512 may include one or more memory buses, as described below. In particular embodiments, one or more memory management units (MMUs) reside between processor 1502 and memory 1504 and facilitate accesses to memory 1504 requested by processor 1502. In particular embodiments, memory 1504 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1504 may include one or more memories 1504, where appropriate. Although this disclosure describes and illustrates particular memory, this disclosure contemplates any suitable memory.

In particular embodiments, storage 1506 includes mass storage for data or instructions. As an example and not by way of limitation, storage 1506 may include a hard disk drive (HDD), a floppy disk drive, flash memory, an optical disc, a magneto-optical disc, magnetic tape, or a Universal Serial Bus (USB) drive or a combination of two or more of these. Storage 1506 may include removable or non-removable (or fixed) media, where appropriate. Storage 1506 may be internal or external to computer system 1500, where appropriate. In particular embodiments, storage 1506 is non-volatile, solid-state memory. In particular embodiments, storage 1506 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1506 taking any suitable physical form. Storage 1506 may include one or more storage control units facilitating communication between processor 1502 and storage 1506, where appropriate. Where appropriate, storage 1506 may include one or more storages 1506. Although this disclosure describes and illustrates particular storage, this disclosure contemplates any suitable storage.

In particular embodiments, I/O interface 1508 includes hardware, software, or both, providing one or more interfaces for communication between computer system 1500 and one or more I/O devices. Computer system 1500 may include one or more of these I/O devices, where appropriate. One or more of these I/O devices may enable communication between a person and computer system 1500. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1508 for them. Where appropriate, I/O interface 1508 may include one or more device or software drivers enabling processor 1502 to drive one or more of these I/O devices. I/O interface 1508 may include one or more I/O interfaces 1508, where appropriate. Although this disclosure describes and illustrates a particular I/O interface, this disclosure contemplates any suitable I/O interface.

In particular embodiments, communication interface 1510 includes hardware, software, or both providing one or more interfaces for communication (such as, for example, packet-based communication) between computer system 1500 and one or more other computer systems 1500 or one or more networks. As an example and not by way of limitation, communication interface 1510 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1510 for it. As an example and not by way of limitation, computer system 1500 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1500 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1500 may include any suitable communication interface 1510 for any of these networks, where appropriate. Communication interface 1510 may include one or more communication interfaces 1510, where appropriate. Although this disclosure describes and illustrates a particular communication interface, this disclosure contemplates any suitable communication interface.

In particular embodiments, bus 1512 includes hardware, software, or both coupling components of computer system 1500 to each other. As an example and not by way of limitation, bus 1512 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1512 may include one or more buses 1512, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

### EMBODIMENTS

Various embodiments may include:
Embodiment 1: A computer-implemented method for searching one or more whole slide images comprising: indexing a whole slide image dataset to generate one or more dataset embeddings corresponding to one or more respective regions of one or more whole slide images, wherein each dataset embedding comprises a feature vector mapping the respective region to a feature embedding space; accessing a query image; generating an embedding for the query image, wherein the embedding comprises a feature vector mapping the query image to the feature embedding space; identifying a plurality of result tiles by comparing the embedding for the query image to one or more of the dataset embeddings, wherein the comparison is based on one or more distances between the embedding for the query image and the one or more of the dataset embeddings in the feature embedding space; and generating a user interface comprising a display of the plurality of result tiles.
Embodiment 2: The computer-implemented method of embodiment 1, wherein identifying the one or more result tiles further comprises: identifying a plurality of the dataset embeddings based on the embedding for the query image; and retrieving the one or more respective regions corresponding to the one or more of the dataset embeddings.
Embodiment 3: The computer-implemented method of any of embodiments 1 to 2, wherein identifying the plurality of the dataset embeddings based on the embedding for the query image comprises identifying a plurality of the dataset embeddings that are within a threshold distance of the embedding for the query image in the feature embedding space.
Embodiment 4: The computer-implemented method of any of embodiments 1 to 3, wherein identifying the plurality of the dataset embeddings based on the embedding for the query image comprises identifying a threshold number of the dataset embeddings ordered based on distance to the embedding for the query image in the feature embedding space.
Embodiment 5: The computer-implemented method of any of embodiments 1 to 4, further comprising: receiving a user input corresponding to one or more of the plurality of result tiles, the user input comprising a marking of one or more of the plurality of result tiles; receiving a user input corresponding to a weighting associated with the one or more marked result tiles; generating an object filter based on the one or more marked result tiles and the user input corresponding to the weighting associated with the one or more marked result tiles; augmenting the embedding for the query image based on a representation of the object filter; identifying a second plurality of result tiles by comparing the embedding for the augmented query image to one or more of the dataset embeddings; and updating the user interface to display the second plurality of result tiles.
Embodiment 6: The computer-implemented method of embodiment any one of embodiments 1 to 5, wherein applying the generated object filter to the one or more dataset embeddings comprises comparing the one or more dataset embeddings to the generated object filter in the feature embedding space.
Embodiment 7: The computer-implemented method of any of embodiments 1 to 6, further comprising: receiving, from a user device, a user input to save the generated object filter; and storing the generated object filter in association with a record for one or more users of the user device.
Embodiment 8: The computer-implemented method of any one of embodiments 1 to 7, further comprising: receiving, from a user device, a user input to share the generated object filter with one or more other users; and storing the generated object filter in association with a record for one or more other users.
Embodiment 9: The computer-implemented method of any of embodiments 1 to 8, wherein indexing the whole slide image dataset comprises, for each of a plurality of whole slide images: segmenting the whole slide image into a plurality of tiles; generating, using an embedding network, a feature vector corresponding to each tile of the plurality of tiles that maps the tile to the feature embedding space; and storing the feature vector in association with the corresponding tile and whole slide image.
Embodiment 10: The computer-implemented method of any of embodiments 1 to 9, wherein accessing the query image comprises: receiving the query image from a user device; receiving a resource locator or unique identifier corresponding to the query image; or receiving a specification of a region of a whole slide image.
Embodiment 11: The computer-implemented method of any of embodiments 1 to 10, wherein the query image corresponds to a whole slide image, the method further comprising: indexing the whole slide image corresponding to the query image to generate one or more additional dataset embeddings corresponding to one or more respective regions of the whole slide image, wherein each dataset embedding comprises a feature vector mapping the respective region to a feature embedding space; and adding the whole slide image corresponding to the query image to the whole slide image dataset.
Embodiment 12: The computer-implemented method of any of embodiments 1 to 11, further comprising: receiving a user input corresponding to one or more of the plurality of result tiles, the user input indicating a relevance of the one or more of the plurality of result tiles to the query image; modifying a weighting of the one or more indicated results based on the user input; identifying a second plurality of result tiles by comparing the embedding for the query image to one or more of the dataset embeddings based on the modified weighting; and updating the user interface to display the second plurality of result tiles.
Embodiment 13: The computer-implemented method of any of embodiments 1 to 12, further comprising: receiving a user input corresponding to one or more of the plurality of result tiles, the user input indicating a relevance of the one or more of the plurality of result tiles to the query image; computing an average embedding of the relevant search results; identifying a second plurality of result tiles by comparing the average embedding of the relevant search results to one or more of the dataset embeddings; and updating the user interface to display the second plurality of result tiles.
Embodiment 14: The computer-implemented method of any of embodiments 1 to 13, further comprising: receiving, from a user device, a user input corresponding to a first result tile of the plurality of result tiles; identifying a first whole slide image corresponding to the first result tile; and updating the user interface to display the first whole slide image.
Embodiment 15: The computer-implemented method of any of embodiments 1 to 14, further comprising: identifying metadata associated with the first whole slide image or the first result tile; and including the metadata in the user interface comprising the display of the first whole slide image, wherein the metadata comprises information regarding the first result tile, the first whole slide image, or a source of the first whole slide image.
Embodiment 16: The computer-implemented method of any of embodiments 1 to 15, further comprising: identifying a plurality of whole slide images corresponding to the plurality of result tiles; identifying a plurality of sources of the whole slide images; and updating the user interface to display a report of information corresponding to the plurality of sources of the whole slide images.
Embodiment 17: The computer-implemented method of any of embodiments 1 to 16, wherein the information corresponding to the plurality of sources comprises conditions diagnosed in the plurality of sources or known outcomes associated with the plurality of sources.
Embodiment 18: The computer-implemented method of any of embodiments 1 to 17, further comprising: identifying, based on the plurality of result tiles, a respective location of one or more features captured in the query image in the plurality of whole slide images corresponding to the plurality of result tiles; and updating the user interface to display a report of the identified respective locations.
Embodiment 19: The computer-implemented method of any of embodiments 1 to 18, further comprising: for at least one of the one or more result tiles: generating, based on the respective distance between the query image embedding and the one or more dataset embeddings in the feature embedding space, a heatmap of the corresponding whole slide image; updating the user interface to display the heatmap.
Embodiment 20: A whole slide image search system comprising one or more processors and a memory coupled to the processors comprising instructions executable by the processors, the processors being operable when executing the instructions to: index a whole slide image dataset to generate one or more dataset embeddings corresponding to one or more respective regions of one or more whole slide images, wherein each dataset embedding comprises a feature vector mapping the respective region to a feature embedding space; access a query image; generate an embedding for the query image, wherein the embedding comprises a feature vector mapping the query image to the feature embedding space; identifying a plurality of result tiles by comparing the embedding for the query image to one or more of the dataset embeddings, wherein the comparison is based on one or more distances between the embedding for the query image and the one or more of the dataset embeddings in the feature embedding space; and generate a user interface comprising a display of the plurality of result tiles.
Embodiment 21: The whole slide image search system of embodiment 20, wherein the processors operable when executing instructions to identify the one or more result tiles comprises the processors being further operable when executing instructions to: identify a plurality of the dataset embeddings based on the embedding for the query image; and retrieve the one or more respective regions corresponding to the one or more of the dataset embeddings.
Embodiment 22: The whole slide image search system of any of embodiments 20 to 21, wherein the processors operable when executing instructions to identify the plurality of the dataset embeddings based on the embedding for the query image comprises the processors being further operable when executing instructions to: identify a plurality of the dataset embeddings that are within a threshold distance of the embedding for the query image in the feature embedding space.
Embodiment 23: The whole slide image search system of any of embodiments 20 to 22, wherein the processors operable when executing instructions to identify the plurality of the dataset embeddings based on the embedding for the query image comprises the processors being further operable when executing instructions to: identify a threshold number of the dataset embeddings ordered based on distance to the embedding for the query image in the feature embedding space.
Embodiment 24: The whole slide image search system of any one of embodiments 20 to 23, wherein the processors are further operable when executing the instructions to: receive a user input corresponding to one or more of the plurality of result tiles, the user input comprising a marking of one or more of the plurality of result tiles; receive a user input corresponding to a weighting associated with the one or more marked result tiles; generate an object filter based on the one or more marked result tiles and the user input corresponding to the weighting associated with the one or more marked result tiles; augment the embedding for the query image based on a representation of the object filter; identify a second plurality of result tiles by comparing the embedding for the augmented query image to one or more of the dataset embeddings; and update the user interface to display the second plurality of result tiles.
Embodiment 25: The whole slide image search system of any of embodiments 20 to 24, wherein the processors operable when executing instructions to apply the generated object filter to the one or more dataset embeddings comprises the processors being further operable when executing instructions to: compare the one or more dataset embeddings to the generated object filter in the feature embedding space.
Embodiment 26: The whole slide image search system of any of embodiments 20 to 25, wherein the processors are further operable when executing the instructions to: receive, from a user device, a user input to save the generated object filter; and store the generated object filter in association with a record for one or more users of the user device.
Embodiment 27: The whole slide image search system of any of embodiments 20 to 26, wherein the processors are further operable when executing the instructions to: receive, from a user device, a user input to share the generated object filter with one or more other users; and store the generated object filter in association with a record for one or more other users.
Embodiment 28: The whole slide image search system of any of embodiments 20 to 27, wherein the processors operable when executing instructions to index the whole slide image dataset comprises the processors being further operable when executing instructions to: for each of a plurality of whole slide images: segment the whole slide image into a plurality of tiles; generate, using an embedding network, a feature vector corresponding to each tile of the plurality of tiles that maps the tile to the feature embedding space; and store the feature vector in association with the corresponding tile and whole slide image.
Embodiment 29: The whole slide image search system of any of embodiments 20 to 28, wherein the processors operable when executing instructions to access the query image comprises the processors being further operable when executing instructions to: receive the query image from a user device; receive a resource locator or unique identifier corresponding to the query image; or receive a specification of a region of a whole slide image.
Embodiment 30: The whole slide image search system of any of embodiments 20 to 29, wherein the query image corresponds to a whole slide image and the processors are further operable when executing the instructions to: index the whole slide image corresponding to the query image to generate one or more additional dataset embeddings corresponding to one or more respective regions of the whole slide image, wherein each dataset embedding comprises a feature vector mapping the respective region to a feature embedding space; and add the whole slide image corresponding to the query image to the whole slide image dataset.
Embodiment 31: The whole slide image search system of any of embodiments 20 to 30, wherein the processors are further operable when executing the instructions to: modify a weighting of the one or more indicated results based on the user input; identify a second plurality of result tiles by comparing the embedding for the query image to one or more of the dataset embeddings based on the modified weighting; and update the user interface to display the second plurality of result tiles.
Embodiment 32: The whole slide image search system of any of embodiments 20 to 31, wherein the processors are further operable when executing the instructions to: receive a user input corresponding to one or more of the plurality of result tiles, the user input indicating a relevance of the one or more of the plurality of result tiles to the query image; compute an average embedding of the relevant search results; identify a second plurality of result tiles by comparing the average embedding of the relevant search results to one or more of the dataset embeddings; and update the user interface to display the second plurality of result tiles.
Embodiment 33: The whole slide image search system of any of embodiments 20 to 32, wherein the processors are further operable when executing the instructions to: receive, from a user device, a user input corresponding to a first result tile of the plurality of result tiles; identify a first whole slide image corresponding to the first result tile; and update the user interface to display the first whole slide image.
Embodiment 34: The whole slide image search system of any of embodiments 20 to 33, wherein the processors are further operable when executing the instructions to: identify metadata associated with the first whole slide image or the first result tile; and include the metadata in the user interface comprising the display of the first whole slide image, wherein the metadata comprises information regarding the first result tile, the first whole slide image, or a source of the first whole slide image.
Embodiment 35: The whole slide image search system of any of embodiments 20 to 34, wherein the processors are further operable when executing the instructions to: identify a plurality of whole slide images corresponding to the plurality of result tiles; identify a plurality of sources of the whole slide images; and update the user interface to display a report of information corresponding to the plurality of sources of the whole slide images.
Embodiment 36: The whole slide image search system of any of embodiments 20 to 35, wherein the information corresponding to the plurality of sources comprises conditions diagnosed in the plurality of sources or known outcomes associated with the plurality of sources.
Embodiment 37: The whole slide image search system of any one of embodiments 20-36, wherein the processors are further operable when executing the instructions to: identify, based on the plurality of result tiles, a respective location of one or more features captured in the query image in the plurality of whole slide images corresponding to the plurality of result tiles; and update the user interface to display a report of the identified respective locations.
Embodiment 38: The whole slide image search system of any one of embodiments 20-37, wherein the processors are further operable when executing the instructions to: for at least one of the one or more result tiles: generate, based on the respective distance between the query image embedding and the one or more dataset embeddings in the feature embedding space, a heatmap of the corresponding whole slide image; and update the user interface to display the heatmap.
Embodiment 39: One or more computer-readable non-transitory storage media embodying software comprising instructions operable when executed to: index a whole slide image dataset to generate one or more dataset embeddings corresponding to one or more respective regions of one or more whole slide images, wherein each dataset embedding comprises a feature vector mapping the respective region to a feature embedding space; access a query image; generate an embedding for the query image, wherein the embedding comprises a feature vector mapping the query image to the feature embedding space; identifying a plurality of result tiles by comparing the embedding for the query image to one or more of the dataset embeddings, wherein the comparison is based on one or more distances between the embedding for the query image and the one or more of the dataset embeddings in the feature embedding space; and generate a user interface comprising a display of the plurality of result tiles.
Embodiment 40: The computer-readable non-transitory storage media of embodiment 39, wherein the software operable when executed to identify the one or more result tiles comprises the software being further operable when executed to: identify a plurality of the dataset embeddings based on the embedding for the query image; and retrieve the one or more respective regions corresponding to the one or more of the dataset embeddings.
Embodiment 41: The computer-readable non-transitory storage media of any of embodiments 39 to 40, wherein the software operable when executed to identify the plurality of the dataset embeddings based on the embedding for the query image comprises the software being further operable when executed to: identify a plurality of the dataset embeddings that are within a threshold distance of the embedding for the query image in the feature embedding space.
Embodiment 42: The computer-readable non-transitory storage media of any of embodiments 39 to 41, wherein the software operable when executed to identify the plurality of the dataset embeddings based on the embedding for the query image comprises the software being further operable when executed to: identify a threshold number of the dataset embeddings ordered based on distance to the embedding for the query image in the feature embedding space.
Embodiment 43: The computer-readable non-transitory storage media of any one of embodiments 39 to 42, wherein the software is further operable when executed to: receive a user input corresponding to one or more of the plurality of result tiles, the user input comprising a marking of one or more of the plurality of result tiles; receive a user input corresponding to a weighting associated with the one or more marked result tiles; generate an object filter based on the one or more marked result tiles and the user input corresponding to the weighting associated with the one or more marked result tiles; augment the embedding for the query image based on a representation of the object filter; identify a second plurality of result tiles by comparing the embedding for the augmented query image to one or more of the dataset embeddings; and update the user interface to display the second plurality of result tiles.
Embodiment 44: The computer-readable non-transitory storage media of any of embodiments 39 to 43, wherein the software operable when executed to apply the generated object filter to the one or more dataset embeddings comprises the software being further operable when executed to: compare the one or more dataset embeddings to the generated object filter in the feature embedding space.
Embodiment 45: The computer-readable non-transitory storage media of any of embodiments 39 to 44, wherein the software is further operable when executed to: receive, from a user device, a user input to save the generated object filter; and store the generated object filter in association with a record for one or more users of the user device.
Embodiment 46: The computer-readable non-transitory storage media of any of embodiments 39 to 45, wherein the software is further operable when executed to: receive, from a user device, a user input to share the generated object filter with one or more other users; and store the generated object filter in association with a record for one or more other users.
Embodiment 47: The computer-readable non-transitory storage media of any of embodiments 39 to 46, wherein the software operable when executed to index the whole slide image dataset comprises the software being further operable when executed to: for each of a plurality of whole slide images: segment the whole slide image into a plurality of tiles; generate, using an embedding network, a feature vector corresponding to each tile of the plurality of tiles that maps the tile to the feature embedding space; and store the feature vector in association with the corresponding tile and whole slide image.
Embodiment 48: The computer-readable non-transitory storage media of any of embodiments 39 to 47, wherein the software operable when executed to access the query image comprises the software being further operable when executed to: receive the query image from a user device; receive a resource locator or unique identifier corresponding to the query image; or receive a specification of a region of a whole slide image.
Embodiment 49: The computer-readable non-transitory storage media of any of embodiments 39 to 48, wherein the query image corresponds to a whole slide image and the software is further operable when executed to: index the whole slide image corresponding to the query image to generate one or more additional dataset embeddings corresponding to one or more respective regions of the whole slide image, wherein each dataset embedding comprises a feature vector mapping the respective region to a feature embedding space; and add the whole slide image corresponding to the query image to the whole slide image dataset.
Embodiment 50: The computer-readable non-transitory storage media of any of embodiments 39 to 49, wherein the software is further operable when executed to: modify a weighting of the one or more indicated results based on the user input; identify a second plurality of result tiles by comparing the embedding for the query image to one or more of the dataset embeddings based on the modified weighting; and update the user interface to display the second plurality of result tiles.
Embodiment 51: The computer-readable non-transitory storage media of any of embodiments 39 to 50, wherein the software is further operable when executed to: receive a user input corresponding to one or more of the plurality of result tiles, the user input indicating a relevance of the one or more of the plurality of result tiles to the query image; compute an average embedding of the relevant search results; identify a second plurality of result tiles by comparing the average embedding of the relevant search results to one or more of the dataset embeddings; and update the user interface to display the second plurality of result tiles.
Embodiment 52: The computer-readable non-transitory storage media of any of embodiments 39 to 51, wherein the software is further operable when executed to: receive, from a user device, a user input corresponding to a first result tile of the plurality of result tiles; identify a first whole slide image corresponding to the first result tile; and update the user interface to display the first whole slide image.
Embodiment 53: The computer-readable non-transitory storage media of any of embodiments 39 to 52, wherein the software is further operable when executed to: identify metadata associated with the first whole slide image or the first result tile; and include the metadata in the user interface comprising the display of the first whole slide image, wherein the metadata comprises information regarding the first result tile, the first whole slide image, or a source of the first whole slide image.
Embodiment 54: The computer-readable non-transitory storage media of any of embodiments 39 to 53, wherein the software is further operable when executed to: identify a plurality of whole slide images corresponding to the plurality of result tiles; identify a plurality of sources of the whole slide images; and update the user interface to display a report of information corresponding to the plurality of sources of the whole slide images.
Embodiment 55: The computer-readable non-transitory storage media of any of embodiments 39 to 54, wherein the information corresponding to the plurality of sources comprises conditions diagnosed in the plurality of sources or known outcomes associated with the plurality of sources.
Embodiment 56: The computer-readable non-transitory storage media of any one of embodiments 39-55, wherein the software is further operable when executed to: identify, based on the plurality of result tiles, a respective location of one or more features captured in the query image in the plurality of whole slide images corresponding to the plurality of result tiles; and update the user interface to display a report of the identified respective locations.
Embodiment 57: The computer-readable non-transitory storage media of any one of embodiments 37-56, wherein the software is further operable when executed to: for at least one of the one or more result tiles: generate, based on the respective distance between the query image embedding and the one or more dataset embeddings in the feature embedding space, a heatmap of the corresponding whole slide image; and update the user interface to display the heatmap.

What is claimed is:

1. A computer-implemented method comprising:
indexing a whole slide image dataset to generate one or more dataset embeddings corresponding to one or more respective regions of one or more whole slide images, wherein each dataset embedding comprises a feature vector mapping the respective region to a feature embedding space;
accessing a query image;
generating an embedding for the query image, wherein the embedding comprises a feature vector mapping the query image to the feature embedding space;
identifying a plurality of result tiles by comparing the embedding for the query image to one or more of the dataset embeddings, wherein the comparison is based on one or more distances between the embedding for the query image and the one or more of the dataset embeddings in the feature embedding space; and
generating a user interface comprising a display of the plurality of result tiles.

2. The computer-implemented method of claim 1, wherein identifying the plurality of result tiles comprises:
identifying a plurality of the dataset embeddings based on the embedding for the query image; and
retrieving the one or more respective regions corresponding to the one or more of the dataset embeddings.

3. The computer-implemented method of claim 2, wherein identifying the plurality of the dataset embeddings based on the embedding for the query image comprises identifying a plurality of the dataset embeddings that are within a threshold distance of the embedding for the query image in the feature embedding space.

4. The computer-implemented method of claim 2, wherein identifying the plurality of the dataset embeddings based on the embedding for the query image comprises identifying a threshold number of the dataset embeddings ordered based on distance to the embedding for the query image in the feature embedding space.

5. The computer-implemented method of claim 2, further comprising:
receiving a user input corresponding to one or more of the plurality of result tiles, the user input comprising a marking of one or more of the plurality of result tiles;
receiving a user input corresponding to a weighting associated with the one or more marked result tiles;
generating an object filter based on the one or more marked result tiles and the user input corresponding to the weighting associated with the one or more marked result tiles;
augmenting the embedding for the query image based on a representation of the object filter;
identifying a second plurality of result tiles by comparing the embedding for the augmented query image to one or more of the dataset embeddings; and
updating the user interface to display the second plurality of result tiles.

6. The computer-implemented method of claim 5, wherein applying the generated object filter to the one or more dataset embeddings comprises comparing the one or more dataset embeddings to the generated object filter in the feature embedding space.

7. The computer-implemented method of claim 5, further comprising:
receiving, from a user device, a user input to save the generated object filter; and
storing the generated object filter in association with a record for one or more users of the user device.

8. The computer-implemented method of claim 5, further comprising:
receiving, from a user device, a user input to share the generated object filter with one or more other users; and
storing the generated object filter in association with a record for one or more other users.

9. The computer-implemented method of claim 1, wherein indexing the whole slide image dataset comprises, for each of a plurality of whole slide images:
segmenting the whole slide image into a plurality of tiles;
generating, using an embedding network, a feature vector corresponding to each tile of the plurality of tiles that maps the tile to the feature embedding space; and
storing the feature vector in association with the corresponding tile and whole slide image.

10. The computer-implemented method of claim 1, wherein accessing the query image comprises:
receiving the query image from a user device;
receiving a resource locator or unique identifier corresponding to the query image; or
receiving a specification of a region of a whole slide image.

11. The computer-implemented method of claim 1, wherein the query image corresponds to a whole slide image, the method further comprising:
indexing the whole slide image corresponding to the query image to generate one or more additional dataset embeddings corresponding to one or more respective regions of the whole slide image, wherein each dataset embedding comprises a feature vector mapping the respective region to a feature embedding space; and
adding the whole slide image corresponding to the query image to the whole slide image dataset.

12. The computer-implemented method of claim 1 further comprising:
receiving a user input corresponding to one or more of the plurality of result tiles, the user input indicating a relevance of the one or more of the plurality of result tiles to the query image;
modifying a weighting of the one or more indicated results based on the user input;
identifying a second plurality of result tiles by comparing the embedding for the query image to one or more of the dataset embeddings based on the modified weighting; and
updating the user interface to display the second plurality of result tiles.

13. The computer-implemented method of claim 1, further comprising:
receiving a user input corresponding to one or more of the plurality of result tiles, the user input indicating a relevance of the one or more of the plurality of result tiles to the query image;
computing an average embedding of the relevant search results;
identifying a second plurality of result tiles by comparing the average embedding of the relevant search results to one or more of the dataset embeddings; and
updating the user interface to display the second plurality of result tiles.

14. The computer-implemented method of claim 1, further comprising:
receiving, from a user device, a user input corresponding to a first result tile of the plurality of result tiles;
identifying a first whole slide image corresponding to the first result tile; and
updating the user interface to display the first whole slide image.

15. The computer-implemented method of claim 14, further comprising:
identifying metadata associated with the first whole slide image or the first result tile; and
including the metadata in the user interface comprising the display of the first whole slide image, wherein the metadata comprises information regarding the first result tile, the first whole slide image, or a source of the first whole slide image.

16. The computer-implemented method of claim 1, further comprising:
identifying a plurality of whole slide images corresponding to the plurality of result tiles;
identifying a plurality of sources of the whole slide images; and
updating the user interface to display a report of information corresponding to the plurality of sources of the whole slide images.

17. The computer-implemented method of claim 16, wherein the information corresponding to the plurality of sources comprises conditions diagnosed in the plurality of sources or known outcomes associated with the plurality of sources.

18. The computer-implemented method of claim 1, further comprising:
identifying, based on the plurality of result tiles, a respective location of one or more features captured in the query image in the plurality of whole slide images corresponding to the plurality of result tiles; and
updating the user interface to display a report of the identified respective locations.

19. A whole slide image search system comprising:
one or more processors; and
one or more computer-readable non-transitory storage media coupled to one or more of the processors and comprising instructions operable when executed by one or more of the processors to cause the system to perform operations comprising:
indexing a whole slide image dataset to generate one or more dataset embeddings corresponding to one or more respective regions of one or more whole slide images, wherein each dataset embedding comprises a feature vector mapping the respective region to a feature embedding space;
accessing a query image;
generating an embedding for the query image, wherein the embedding comprises a feature vector mapping the query image to the feature embedding space;
identifying a plurality of result tiles by comparing the embedding for the query image to one or more of the dataset embeddings, wherein the comparison is based on a distance between the embedding for the query image and the one or more of the dataset embeddings in the feature embedding space; and
generating a user interface comprising a display of the plurality of result tiles.

20. One or more computer-readable non-transitory storage media including instructions that, when executed by one or more processors, are configured to cause the one or more processors of a whole slide image search system to perform operations comprising:
indexing a whole slide image dataset to generate one or more dataset embeddings corresponding to one or more respective regions of one or more whole slide images, wherein each dataset embedding comprises a feature vector mapping the respective region to a feature embedding space;
accessing a query image;
generating an embedding for the query image, wherein the embedding comprises a feature vector mapping the query image to the feature embedding space;
identifying a plurality of result tiles by comparing the embedding for the query image to one or more of the dataset embeddings, wherein the comparison is based on a distance between the embedding for the query image and the one or more of the dataset embeddings in the feature embedding space; and
generating a user interface comprising a display of the plurality of result tiles.
